(19) United States
(12) Patent Application Publication
(10) Pub. No.: US 2023/0066339 A1
(43) Pub. Date: Mar. 2, 2023

(54) SYSTEMS AND METHODS FOR END-TO-END PET CARE TRANSACTIONS WEB PLATFORM

(71) Applicant: Mars, Incorporated, McLean, VA (US)

(72) Inventors: Christopher Jude Gonzalez, Philadelphia, PA (US); Ryan Charles, New Castle, DE (US)

(21) Appl. No.: 17/822,485

(22) Filed: Aug. 26, 2022

Related U.S. Application Data
(60) Provisional application No. 63/237,731, filed on Aug. 27, 2021.

(51) Int. Cl. G06Q 10/02; G06Q 50/10; G06F 3/0481

(52) U.S. Cl. CPC G06Q 10/02; G06Q 50/10; G06F 3/0481

(57) ABSTRACT

A computer-implemented method for dynamically managing electronic transactions of pet care services data, the method comprising hosting a pet care services portal corresponding to a user and at least one pet, receiving pet service search data, determining one or more external pet care services that match the pet service search data, displaying the one or more external pet care services on a user interface, creating at least one external pet care service reservation in response to a user selection of at least one external pet care service, determining a medical record requirement for the at least one external pet care service, sending at least one medical record request to at least one external pet medical service, receiving at least one medical record associated with the at least one pet from the at least one external pet medical service, and updating the at least one external pet care service reservation.

### CROSS-REFERENCE TO RELATED APPLICATION(S)

This patent application claims the benefit of priority to U.S. Provisional Application No. 63/237,731, filed Aug. 27, 2021, which is incorporated herein by reference in its entirety.

### TECHNICAL FIELD

Various embodiments of this disclosure relate generally to systems and methods for an end-to-end pet care transactions web platform.

### BACKGROUND

A pet owner's journey to discover and book different pet care services is currently a fragmented process that has not kept up with the new generation of pet parents. Current solutions are time consuming, lack provider transparency, lack turn-key resolution, and are not optimized to help pet parents meet their unique criteria. Moreover, many pet care professionals have not adopted technology to help grow and manage their businesses because of the investment required and the lack of “off-the-shelf” options suitable for their needs. For example, no options exist for advanced online scheduling for pet care. Further, conventional techniques fail to provide a single destination for digital scheduling and booking of pet services, and also fail to provide transparency in provider information.

This disclosure is directed to addressing above-referenced challenges. The background description provided herein is for the purpose of generally presenting the context of the disclosure. Unless otherwise indicated herein, the materials described in this section are not admitted to be prior art, or suggestions of the prior art, by inclusion in this section.

### SUMMARY OF THE DISCLOSURE

According to certain aspects of the disclosure, methods and systems are disclosed for an end-to-end pet care transactions web platform.

In one aspect, an exemplary embodiment of a method for dynamically managing electronic transactions of pet care services data. The method may include hosting, by one or more processors, a pet care services portal corresponding to a user and at least one pet, the pet care services portal being based on at least one user profile and at least one pet profile. The method may further include receiving, by the one or more processors, pet service search data input by the user via the pet care services portal. The method may further include, in response to the receiving, determining, by the one or more processors, one or more external pet care services that match the pet service search data. The method may further include displaying, by the one or more processors, the one or more external pet care services on a user interface of the pet care services portal. The method may further include creating, by the one or more processors, at least one external pet care service reservation in response to a user selection of at least one external pet care service. The method may further include, in response to creating the at least one external pet care service reservation, determining, by the one or more processors, a medical record requirement for the at least one external pet care service. The method may further include sending, by the one or more processors, at least one medical record request associated with the at least one pet to at least one external pet medical service, the at least one medical record request indicating the medical record requirement. The method may further include receiving, by the one or more processors, at least one medical record associated with the at least one pet from the at least one external pet medical service. The method may further include updating, by the one or more processors, the at least one external pet care service reservation based on the at least one medical record.

In a further aspect, an exemplary embodiment of a computer system for dynamically managing electronic transactions of pet care services data, the computer system including at least one memory storing instructions, and at least one processor configured to execute the instructions to perform operations. The operations may include hosting a pet care services portal corresponding to a user and at least one pet, the pet care services portal being based on at least one user profile and at least one pet profile. The operations may further include receiving pet service search data input by the user via the pet care services portal. The operations may further include, in response to the receiving, determining one or more external pet care services that match the pet service search data. The operations may further include displaying the one or more external pet care services on a user interface of the pet care services portal. The operations may further include creating at least one external pet care service reservation in response to a user selection of at least one external pet care service. The operations may further include, in response to creating the at least one external pet care service reservation, determining a medical record requirement for the at least one external pet care service. The operations may further include sending at least one medical record request associated with the at least one pet to at least one external pet medical service, the at least one medical record request indicating the medical record requirement. The operations may further include receiving at least one medical record associated with the at least one pet from the at least one external pet medical service. The operations may further include updating the at least one external pet care service reservation based on the at least one medical record.

In a further aspect, an exemplary embodiment of a non-transitory computer-readable medium containing instructions that, when executed by a processor, cause the processor to perform operations for dynamically managing electronic transactions of pet care services data. The operations may include hosting a pet care services portal corresponding to a user and at least one pet, the pet care services portal being based on at least one user profile and at least one pet profile. The operations may further include receiving pet service search data input by the user via the pet care services portal. The operations may further include, in response to the receiving, determining one or more external pet care services that match the pet service search data. The operations may further include displaying the one or more external pet care services on a user interface of the pet care services portal. The operations may further include creating at least one external pet care service reservation in response to a user selection of at least one external pet care service. The operations may further include, in response to creating the at least one external pet care service reservation, determining a medical record requirement for the at least one external pet care service. The operations may further include sending at least one medical record request associated with the at least one pet to at least one external pet medical service, the at least one medical record request indicating the medical record requirement. The operations may further include receiving at least one medical record associated with the at least one pet from the at least one external pet medical service. The operations may further include updating the at least one external pet care service reservation based on the at least one medical record.

It is to be understood that both the foregoing general description and the following detailed description are exemplary and explanatory only and are not restrictive of the disclosed embodiments.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A-C depict an exemplary embodiment of a booking engine that is part of a web platform, according to one or more embodiments.

FIG. 2A-E depict an exemplary embodiment of a booking engine's search and filter functionality, according to one or more embodiments.

FIG. 3 depicts an exemplary embodiment of a booking engine's search result functionality, according to one or more embodiments.

FIG. 4 depicts an exemplary embodiment of a booking engine's search filter functionality, according to one or more embodiments.

FIGS. 5A-C depict an exemplary embodiment of a groomer provider profile page, according to one or more embodiments.

FIGS. 6A-C depict an exemplary embodiment of a veterinary provider profile page, according to one or more embodiments.

FIG. 7 depicts an exemplary embodiment of a trainer provider profile page, according to one or more embodiments.

FIG. 8 depicts an exemplary embodiment of a booking details page, according to one or more embodiments.

FIG. 9 depicts an exemplary embodiment of a booking confirmation page, according to one or more embodiments.

FIG. 10 depicts an exemplary flowchart illustrating actions that may be performed by users of the web platform, according to one or more embodiments.

FIG. 11 depicts an exemplary embodiment of a user profile page, according to one or more embodiments.

FIG. 12A-D depict an exemplary embodiment of a pet profile page, according to one or more embodiments.

FIG. 13 depicts an exemplary embodiment of a pet profile page, according to one or more embodiments.

FIG. 14 depicts a flowchart of an exemplary method for dynamically managing electronic transactions of pet care services data, according to one or more embodiments.

FIG. 15 depicts an exemplary environment that may be utilized with techniques presented herein, according to one or more embodiments.

FIG. 16 depicts an example of a computing device that may execute the techniques described herein, according to one or more embodiments.

### DETAILED DESCRIPTION OF EMBODIMENTS

According to certain aspects of the disclosure, methods and systems are disclosed for an end-to-end pet care transactions web platform.

A user's journey to discover and book different pet care services is currently a fragmented process that has not kept up with the new generation of users. Current solutions are time consuming, lack provider transparency, lack turn-key resolution, and are not optimized to help users meet their unique criteria. For example, users are searching online for recommendations, ranging from veterinarians to groomers to trainers, and are open to suggestions. They want a single destination and crave transparency in provider information, as well as ease of use in digital scheduling and booking.

A solution to such problem involves a singular destination that fulfills all of the needs of a user. More specifically, such solution may involve building a transparent, trustworthy destination for professional pet service needs. Components of such a solution may include a booking engine, transparency in pet service provider selection and information, and filtering functionality to facilitate a fine-tuned search.

The web platform disclosed herein may centralize an array of related, professional pet services into a single, trustworthy destination. Through the web platform, users are able to discover and book a variety of professional pet care services, including grooming, training, veterinary, holistic pet care services, and the like.

The web platform creates a vibrant marketplace with local pet care businesses and users. If a pet needs training, grooming, or a wellness check-up, the web platform may be a single destination to find and book highly skilled professionals for any service. The web platform creates value for providers by simplifying and digitizing tasks, while offering a robust online presence that directs new and repeat customers to the providers.

As will be discussed in more detail below, in various embodiments, systems and methods are described for dynamically managing electronic transactions of pet care services data. The systems and methods may host a pet care services portal corresponding to a user and at least one pet, the pet care services portal being based on at least one user profile and at least one pet profile. The systems and methods may receive pet service search data input by the user via the pet care services portal. The systems and methods may, in response to the receiving, determine one or more external pet care services that match the pet service search data. The systems and methods may display the one or more external pet care services on a user interface of the pet care services portal. The systems and methods may create at least one external pet care service reservation in response to a user selection of at least one external pet care service. The systems and methods may, in response to creating the at least one external pet care service reservation, determine a medical record requirement for the at least one external pet care service. The systems and methods may send at least one medical record request associated with the at least one pet to at least one external pet medical service, the at least one medical record request indicating the medical record requirement. The systems and methods may receive at least one medical record associated with the at least one pet from the at least one external pet medical service. The systems and methods may update the at least one external pet care service reservation based on the at least one medical record.

Reference to any particular activity is provided in this disclosure only for convenience and not intended to limit the disclosure. The disclosure may be understood with reference to the following description and the appended drawings.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, a term such as “user” or the like generally encompasses a pet parent and/or pet parents. A term such as “pet” or the like generally encompasses a user's pet, where the term may encompass multiple pets. A term such as “provider,” “service provider,” or the like generally encompasses a pet care business. A term such as “resource” or the like generally encompasses an employee or employees of a pet care business, where the pet care business may have multiple employees. A term such as “request” or the like generally encompasses a user request for an appointment with a provider. A term such as “appointment” or “booking” or the like generally encompasses a confirmed and/or completed request by a provider. A term such as “records request” or the like generally encompasses a customer requesting records from a veterinarian. A term such as “fulfilled request” or the like generally encompasses a fulfilled records request and/or records attached to a pet profile.

A term such as “non-registered user” or the like generally encompasses a user that has not registered. A term such as “registered user” or the like generally encompasses a user that has registered, created a pet profile, has a customized experience based on profile data, and can complete a booking process with any active provider. A term such as “non-registered provider” or the like generally encompasses a business that has not yet claimed its listing. A term such as “registered provider” or the like generally encompasses a business that has claimed its listing, edited its business information, and can actively receive, confirm, and/or complete user bookings.

### Exemplary Pet Care Services Booking Process

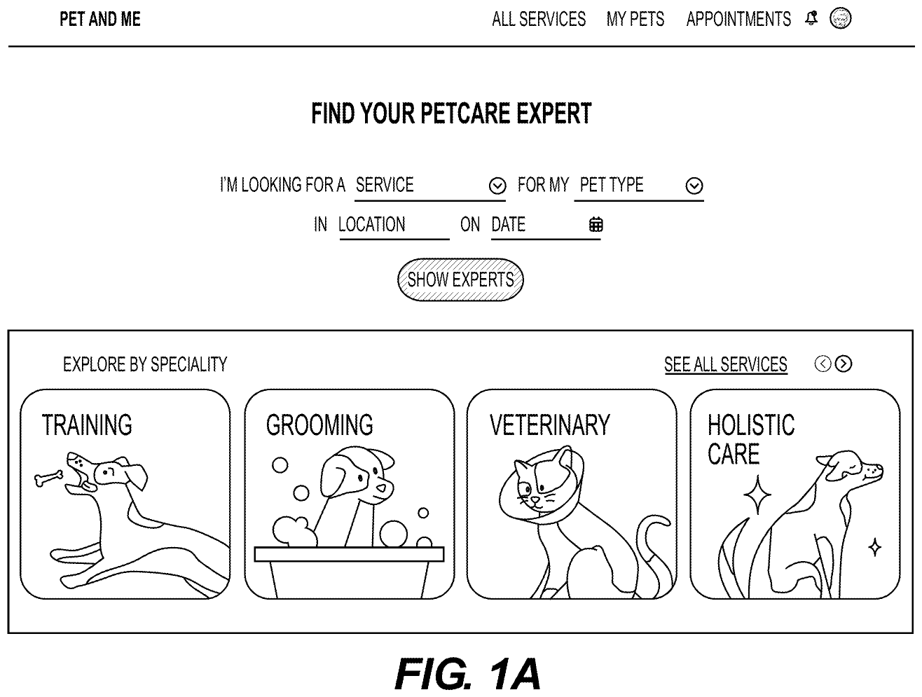

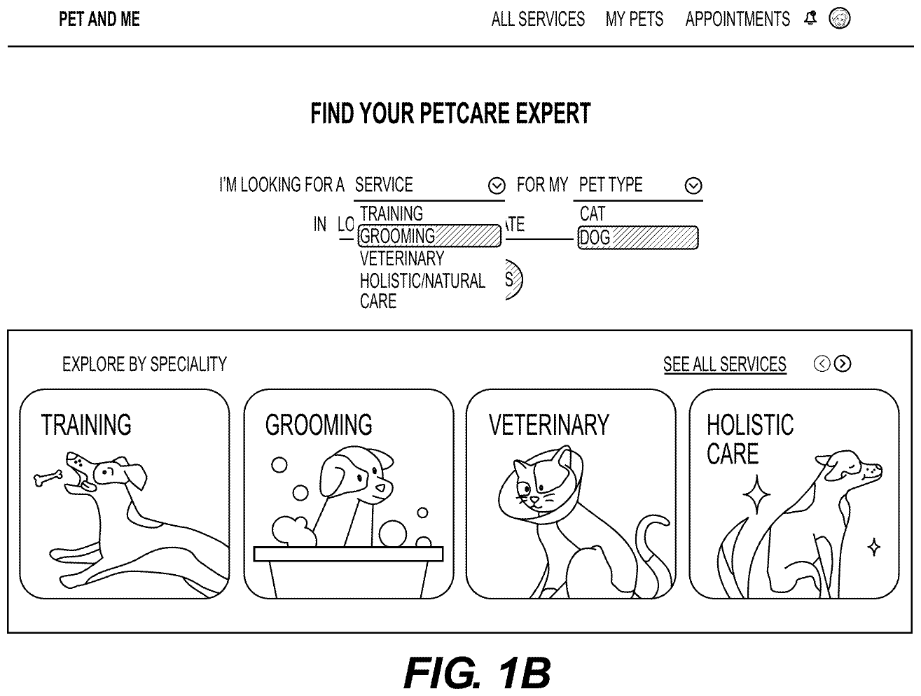

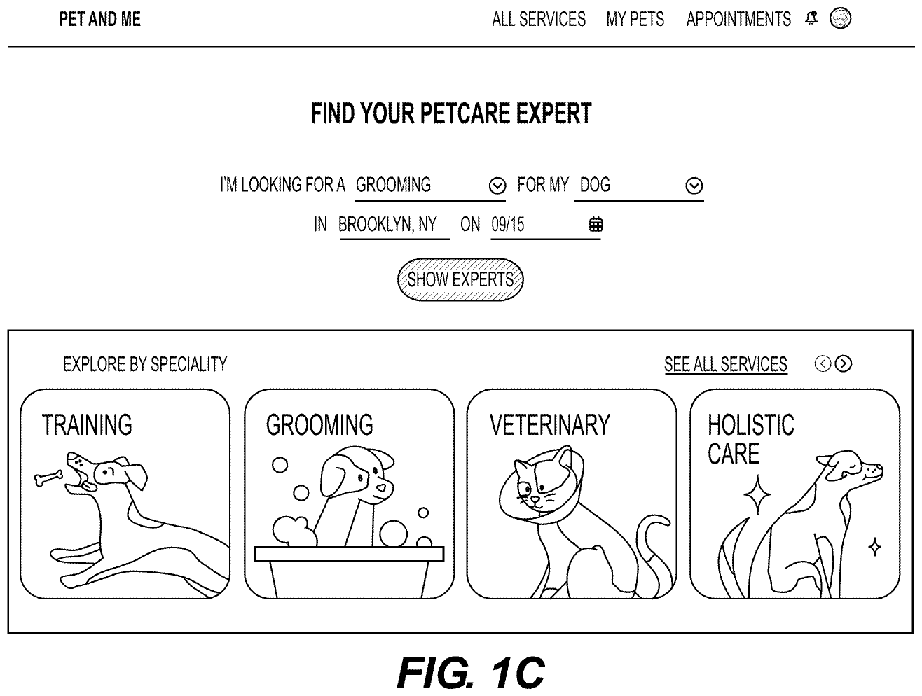

A booking engine that is part of the web platform, as illustrated in FIGS. 1A-C, may be presented for searching for and reserving pet care services, such as training, grooming, veterinary, and holistic care (e.g., nutritionist, naturopathic). The booking engine may allow a user to search for and select from numerous external third party systems, such as: (1) grooming services, (2) training services, (3) veterinary services, and/or (4) holistic services. Service aggregation may provide for the consolidating of multiple pet care service types and providers into the web platform for utilization by the booking engine.

The user may engage with user discovery features to search for one or more pet care services. For example, natural language selection may provide a search function with predetermined choices. For example, as illustrated in FIGS. 1A-C, the user may make selections corresponding to a service, pet type, location, and/or a date. The service selection may include training, grooming, veterinary, and/or holistic care. The grooming service selection may enable users to discover, book, and/or pay for grooming services. The training service selection may enable users to discover, book, and/or pay for training services. The veterinary service selection may enable users to discover, book, and/or pay for veterinary services. The holistic service selection may enable users to discover, book, and/or pay for holistic services. The pet type may include a cat, a dog, and the like. The location may be input by the user or automatically determined by the web platform (e.g., using one or more APIs). The date may include the current day and future days for a period of days, weeks, or years. For example, a calendar may show days from Sunday to Saturday, where available booking dates will be shown for 15 weeks past the closest Saturday.

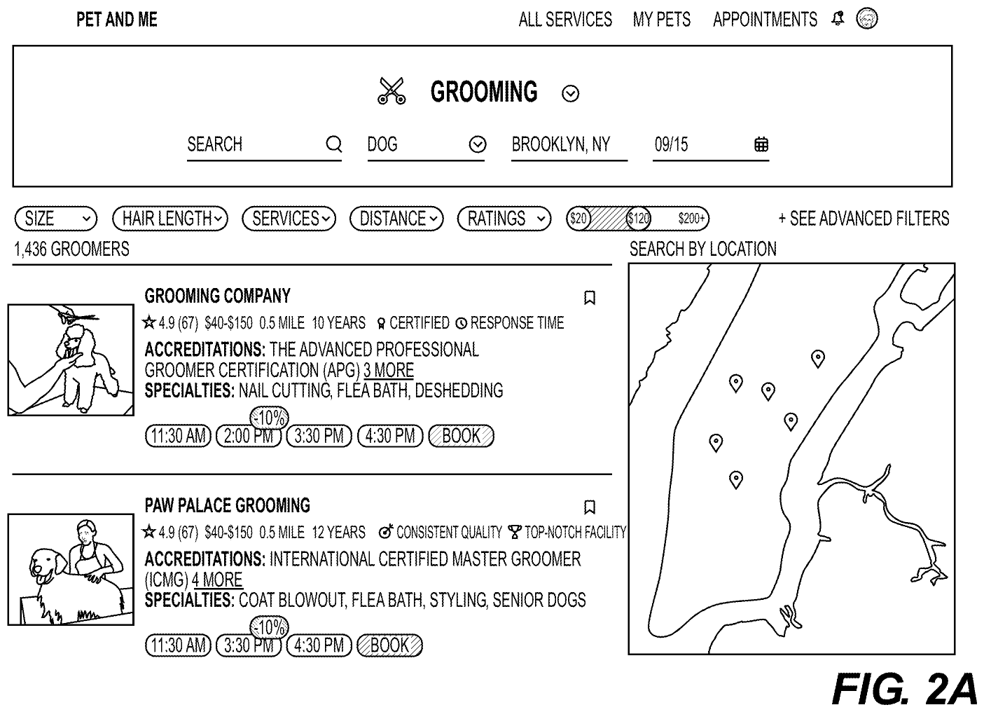

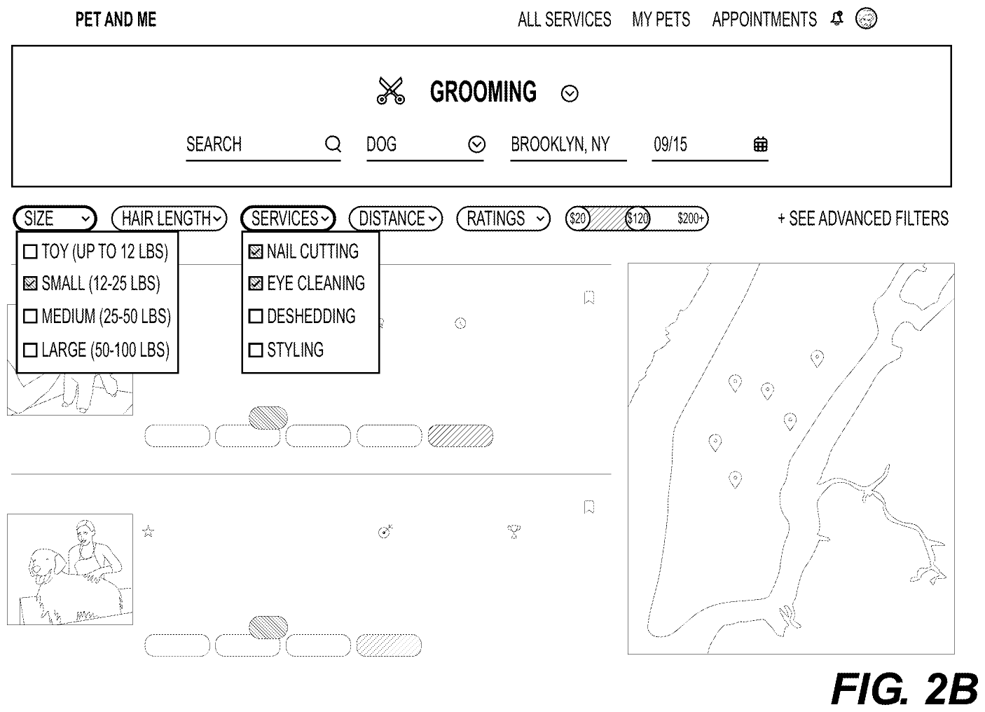

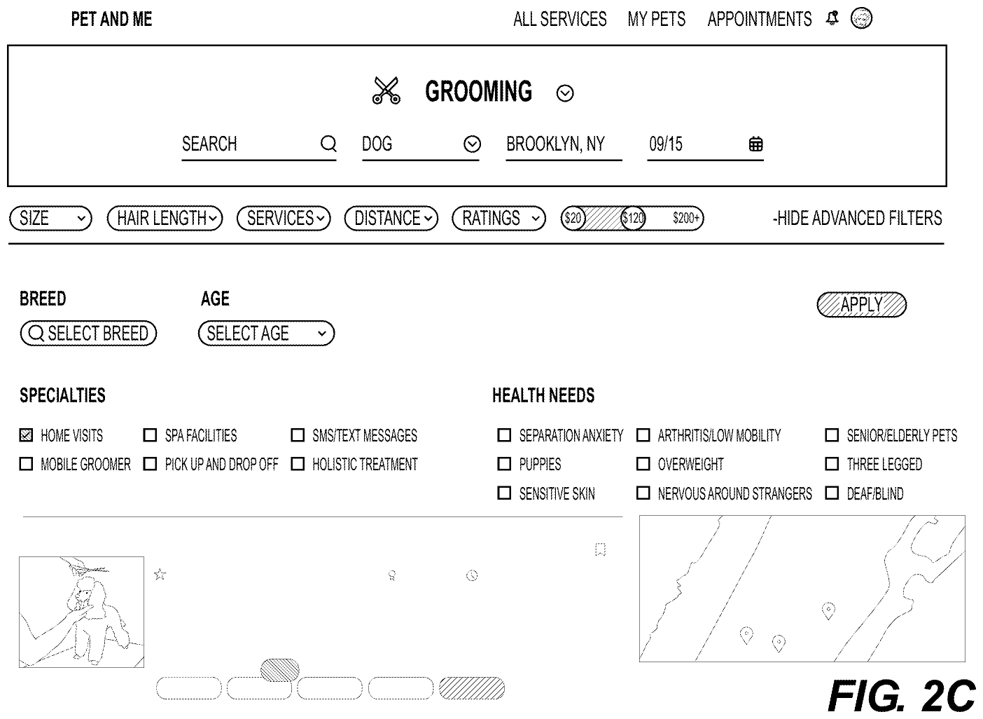

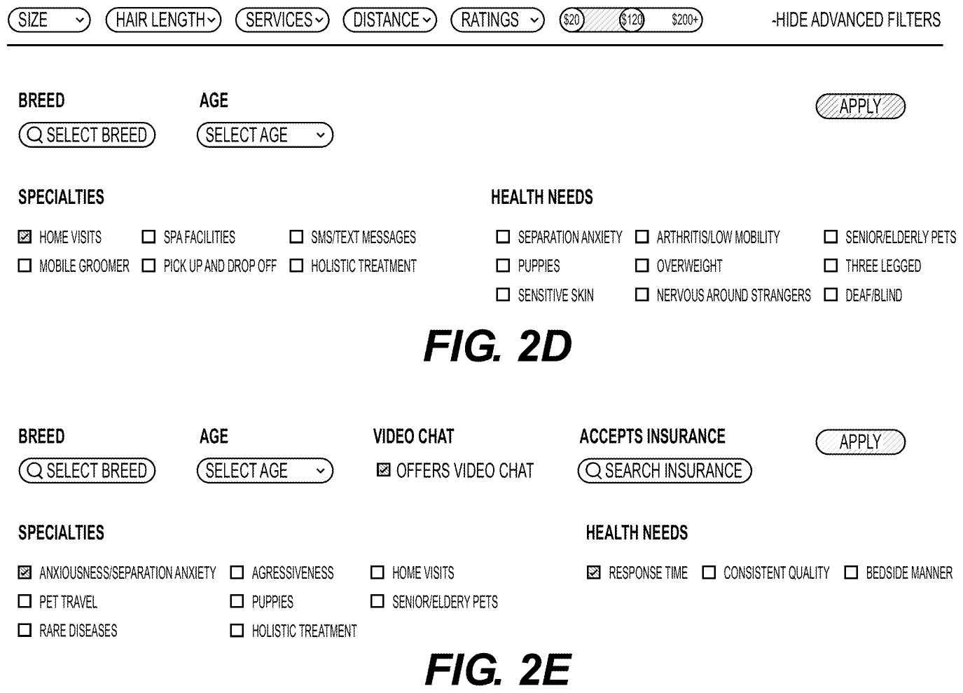

In response to the one or more search selections, the booking engine may display one or more pet care services that match the one or more search selections, as illustrated in FIG. 2A. The search results may be displayed via the web platform on a handheld mobile device, a desktop computer, or any other device. The booking engine may present open dates and time slots, which correspond to the selected service provider, to the user. Additionally, in some embodiments, a map rendering may display nearby search result locations from a user's search. In some embodiments, a broad search for a service type may render relevant default results to the user. The relevant default results may prioritize claimed, active businesses and then prioritize proximity to the input location.

In some embodiments, the providers may include traveling providers that may not have a physical location. For example, traveling grooming/training services may not have a physical location, but may travel to where clients are located. Travel providers may inform the booking engine of a particular distance or area that they will travel to provide services. The booking engine may consider such information when displaying the search results to the user.

The booking engine may display general filters and/or advanced filters that allow the user to filter the one or more pet care services that resulted from the one or more search selections, as illustrated in FIGS. 2B-E, 3, and 4. General filters may include filters that enable users to sort the results by pet size, coat type, services, distance, ratings, and price. The general filters may allow a user to make one or several selections. The pet size may include four categories: (1) toy (up to 12 lbs), (2) small (12-25 lbs), (3) medium (25-50 lbs), and (4) large (greater than 50 lbs). The coat type may correspond to the type of pet coat and/or fur, where the coat type may include five categories: (1) smooth/short, (2) wire-haired, (3) long, (4) curly, and (5) double coated. The services may vary based on the type of service being filtered. For example, if a grooming service is selected, the types of services may include bath and brush-out, deshedding, full service groom, nail clipping, ear cleaning, teeth brushing, and/or anal gland expression. If a training service is selected, the types of services may include virtual, private, and/or group. The distance may include at least one of the following options: less than a mile, between 1 and 5 miles, between 5 and 10 miles, between 10 and 25 miles, and greater than 25 miles. Additionally there may be a cap, where options will not be shown past a certain distance. For example, options may not be shown that are greater than 50 miles away. The ratings may include the following options: high to low (by rating), most reviews (highest reviewed to lowest reviewed), and/or four or more stars. The price may include a slider with a price starting at 20 dollars and going up to 200+ dollars.

The advanced filters may enable additional filtering based on breed, provider offerings, and/or the provider's experience. When a user begins to type the name of a pet breed, the advanced filters may autocomplete the breed name based on a Breed API, which may filter for providers that specialize in a particular dog breed. The provider offerings may provide filters for providers that offer home visits, mobile grooming, spa facilities, and/or transportation (pick up/drop off). The provider's experience filter may allow for users to search for providers who have experience with puppies, separation anxiety, sensitive skin, arthritis/low mobility, nervous around strangers, deaf, blind, overweight, senior/elderly, and/or large breeds.

Other discovery features may include location searching and/or specific business searching. Location searching may prompt the user for zip codes and/or an address, with auto-completion for addresses based on a partial entry from a mapping API. Specific business searching may provide the ability for users to search for a specific business, with auto-completion based on a partial entry from a database of listed providers.

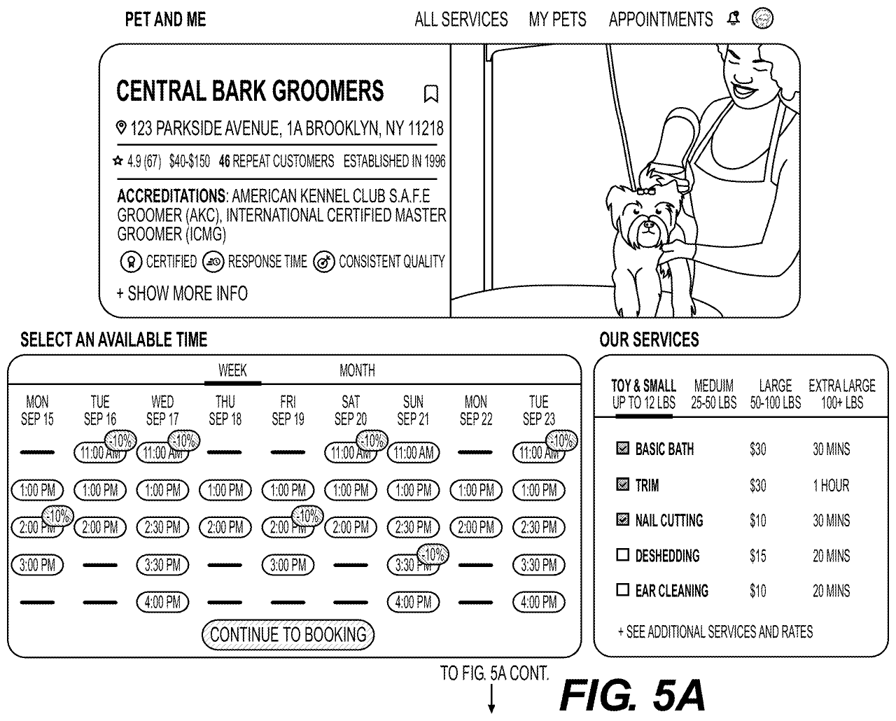

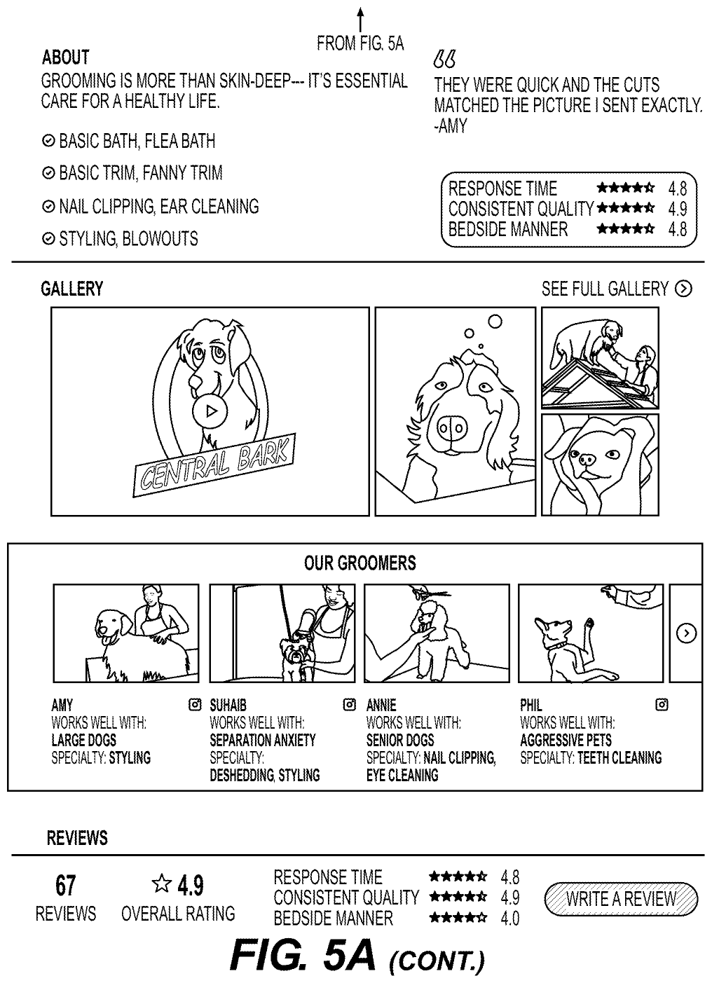

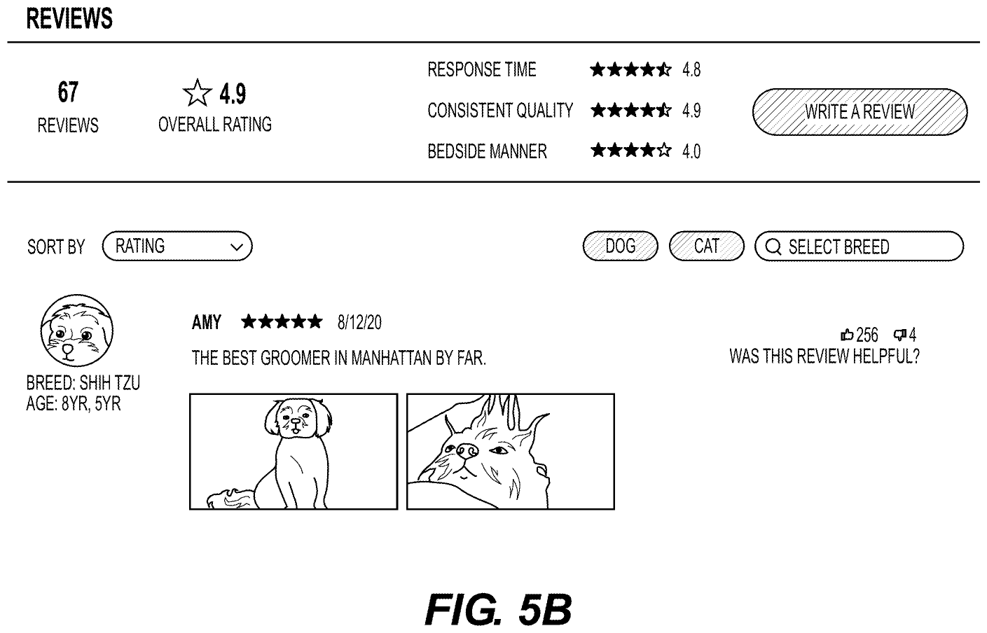

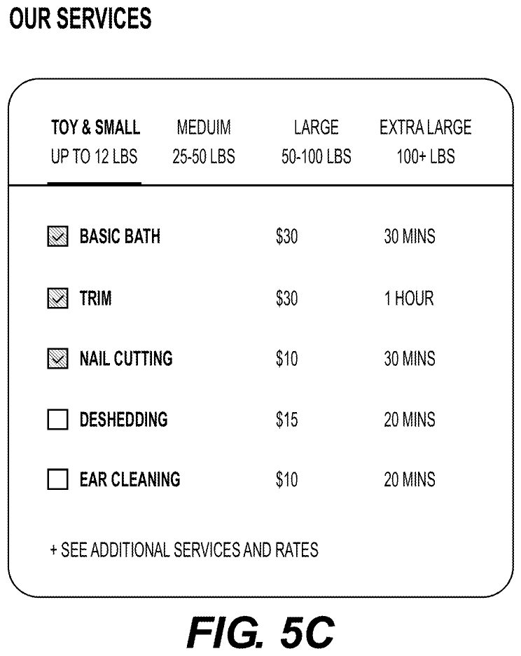

As illustrated in FIGS. 5A-C, 6A-C, and 7, in response to the user selecting a particular pet care service, the booking engine may display a provider profile page. The provider profile page may act as a digital storefront within the web platform, where each provider profile page may correspond to a different provider. Additionally, in some embodiments, the web platform may provide one or more incentives to one or more providers in order to encourage providers to create a provider profile page. Example incentives may include discount rates, complimentary advertising, and the like. The provider profile page may display essential information about the pet care service, such as accreditations, profiles/bios, hours of operation, services offered, individual resource profiles, repeat customers, links to corresponding social media profiles, a media gallery, and/or reviews. The accreditations may be specific to each service and may include the state certification, the number of years of experience and/or the number of years in the business, training and/or educational certificates, insurance, and/or specialties. Such specialties may include specialties in dealing with aggression, house training, arthritis, and/or nutrition. Additionally, for example, the profiles/bios may include information about the business and bios for each resource. If the business is a sole proprietorship, there may be a singular “about” the business. The profile/bio description may include how long the provider has been in business. Additionally, the profiles/bios may be written in free form, where there may be a character limit (such as 1000 characters). The services offered may include a tiered menu of the services offered by the provider. The individual resource profiles may include a picture of one or many employees, as well as a “Works well With” section, an “Experience with” section, and/or a “Favorite Breeds” section. The “Works well With” section may include checkboxes associated with the “Experience With” section. The “Favorite Breeds” section may allow the provider to select one or many breeds, where the breeds may be autocompleted based on the Breed API. The repeat customers section may show if the provider has repeat customers, where such information may be based on internal bookings.

A reviews and ratings system may provide users with the ability to leave providers a numerical 1-5 rating, such as in stars, with 5 being the highest possible rating, and/or a text review. The text review may include a minimum and a maximum number of characters. For example, the text review may include a minimum of 240 characters and a maximum of 5000 characters. Providers may have the ability to respond to reviews. Such responses may include the same character limitations. Additionally, a provider's average rating and/or a number of ratings may be displayed in the search results. The review and rating system may include subratings that may cover the condition of the facility, the bedside manner of the workers, and how long previous customers had to wait for the service.

The provider profile page may allow the user to select a date and/or time of a service and/or the particular service(s) the user would like to book. For example, the user may select a date and/or time from the displayed open dates and time slots of a matching service. The user may also have the option to select one or more services that the user would like book. Upon selecting a date and/or time, the booking may be confirmed and the user may receive an instant confirmation of booking, such as via text or email.

Providers, via the provider profile page, may also be able to easily set future availability and/or capacity for appointments, including specific resources and/or employee availability, confirm/edit/cancel appointment requests, and/or maintain/update their business information and offerings. The providers may also include a particular capacity during the available times. For example, a groomer may have availability to groom four pets between 9 am and 11 am, but the same groomer may have availability to groom only two pets between 1 pm and 4 pm.

For example, providers may be able to set a “regular” schedule, which may be a schedule that sets the provider's default availability into the future. For example, if a resource and/or employee works 9 am to 5 pm Monday through Friday, the provider can set such schedule as the employee's default availability. The provider may only need to update/change the employee's default availability when the employee has vacation days or other one-off changes to the set schedule.

Providers may also have the ability to set constraints or limits on the amount of bookings that the provider can take at specific times. For example, a trainer offering a group class may set the class limit at 5, allowing up to five users to book such class. Additionally, for example, a groomer often books clients in overlapping time slots so that while they wait for a dog to dry after a bath, the groomer may move on to another client to groom them.

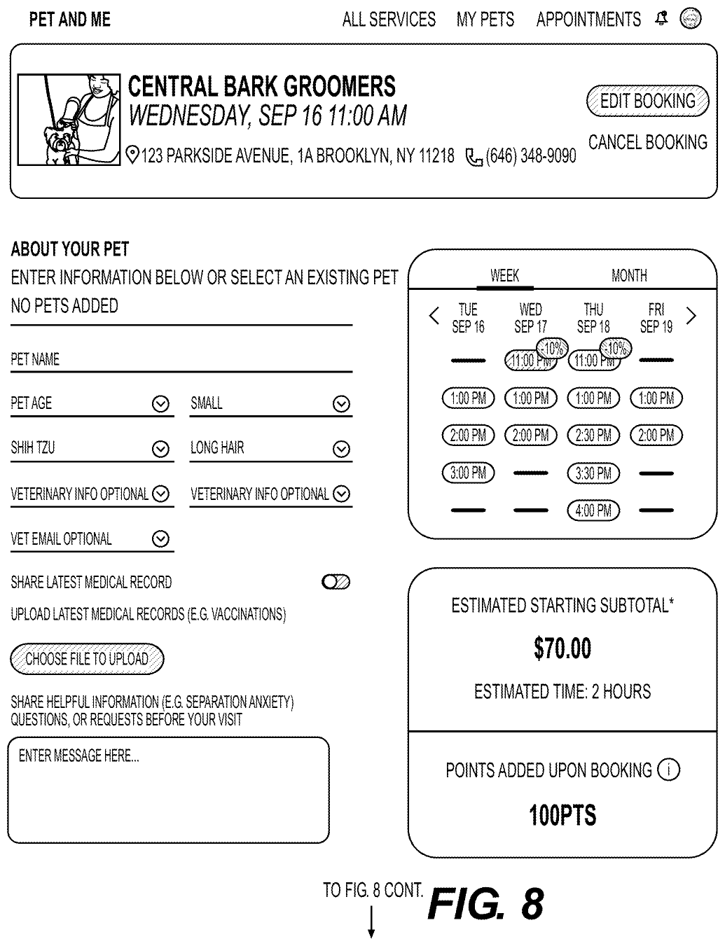

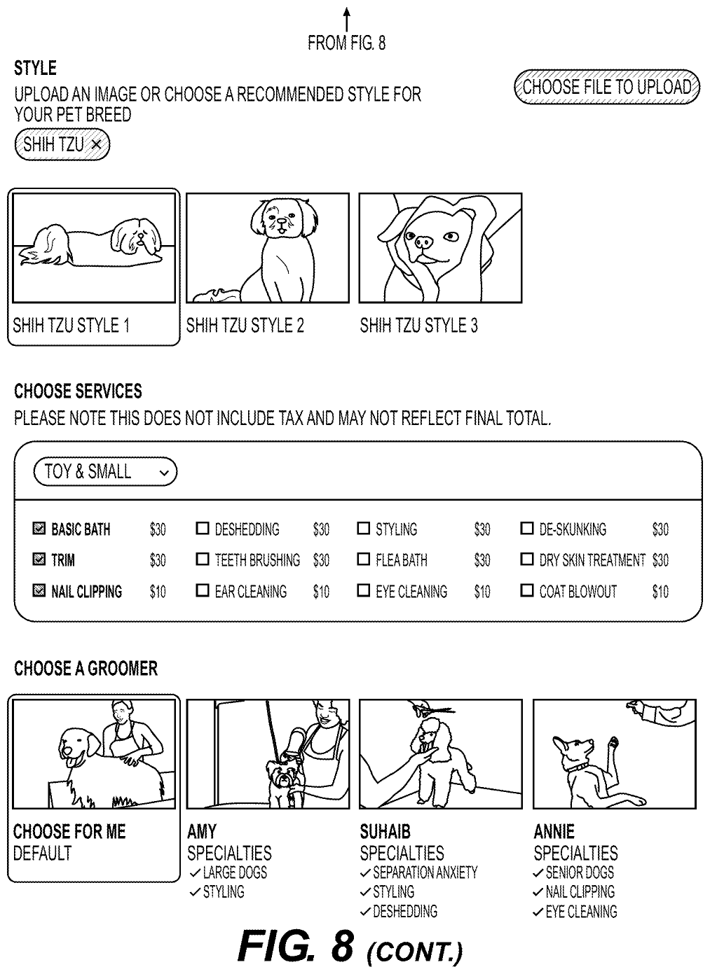

The booking engine may also display a booking details page, as illustrated in FIG. 8. The booking details page may include an interactive booking calendar that may include an interface to provide customers to find available time slots and request an appointment. Times may be displayed in multi-hour windows for users (e.g., 9 am-noon, noon-3 pm, 3 pm-6 pm, 6 pm-9 pm). Unavailable days or times may be greyed out and may not available to be selected. The booking details page may include an open form for users to write special requests or notes to their service provider before, during, and/or after the booking process. The booking details page may include a services menu selection form that may provide options for users to select (such as via a check box) specific services that they want to include for their requested appointment. The booking details page may also provide the ability for the user to include the pet's details, such as name, age, size, breed, hair length, veterinarian information, and/or medical records. The booking details page may provide the option for the user to select a particular employee that would perform the service (e.g., “choose a groomer”). The booking details page may allow the user to send a message to the service provider. For example, the user may send a message that the pet has a limp. The booking details page may allow a user to upload one or more pictures. For example, a media gallery may allow providers to upload photos and/or videos showcasing their facilities, services/results, etc., in order to build their business page and profile. A profile photo may be chosen by the business that will be the provider's identifier when displayed in the search results. The booking details page may provide one or more images corresponding to options for the services. For example, the booking details page may display one or more hairstyle options that the user may select from. The user may also have the ability to instantly pay for the booked service. For example, the booking details page may include the ability to process payments via debit card, credit card, Venmo, PayPal, promo code, and the like at the time of booking. Alternatively, the user may be able to pay upon the finalization of the service(s). The booking details page may also present an incentive discount for selecting a low volume timeslot. For example, the booking details page may present a discount of five dollars for a grooming that takes place at 2 pm on a Tuesday. Additionally, there may be a loyalty program, such as a point system, for using the booking engine. For example, the booking details page may include points that are redeemable towards free services of the service provider/business. The booking details page may include a recurring appointments feature that may provide the ability for users to request recurring appointments based on defined time interval (e.g., every 4 weeks, or every 3 months).

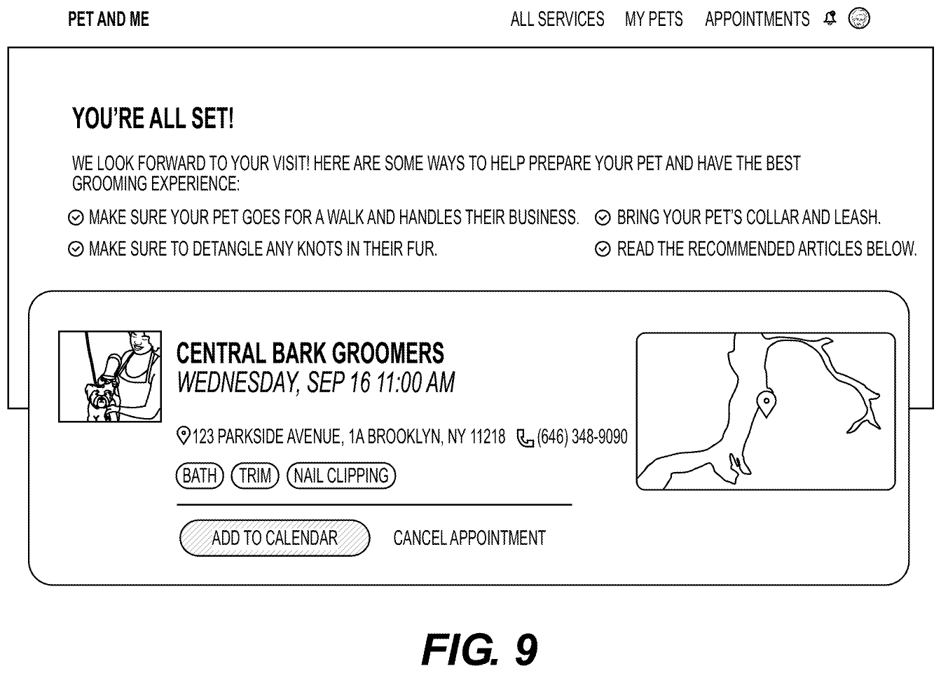

Once a booking is confirmed (e.g., a user fills out and submits the booking details form), an “add to calendar” option may be displayed to the user, as illustrated in FIG. 9. The “add to calendar” option may provide the ability for the user to add the booking, as well as booking details, to a user's personal calendar. Additionally, an edit/cancel appointments feature may provide the ability for users to edit an existing booking or cancel an existing booking.

Upon the completion of the booked service, a post-service report card may be sent to the user with a link to review the service provider and/or give the service provider a tip. Additionally, when the user logs back in, if the user has not reviewed the service provider, the web platform may prompt the user to review the service provider. However, the user may be allowed to navigate away from the prompt without reviewing the provider.

### Exemplary Web Platform Process

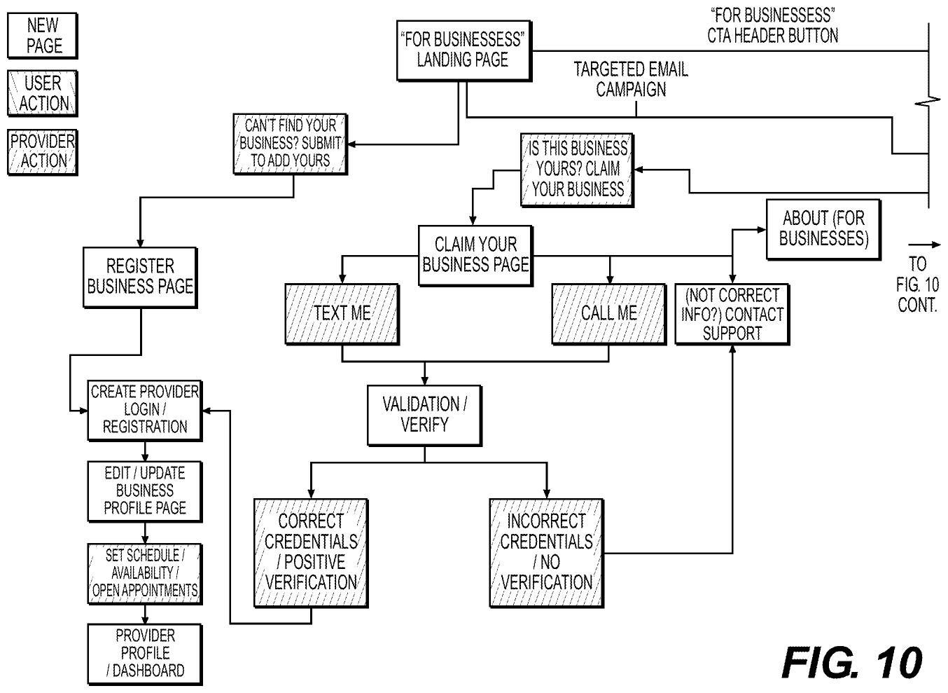

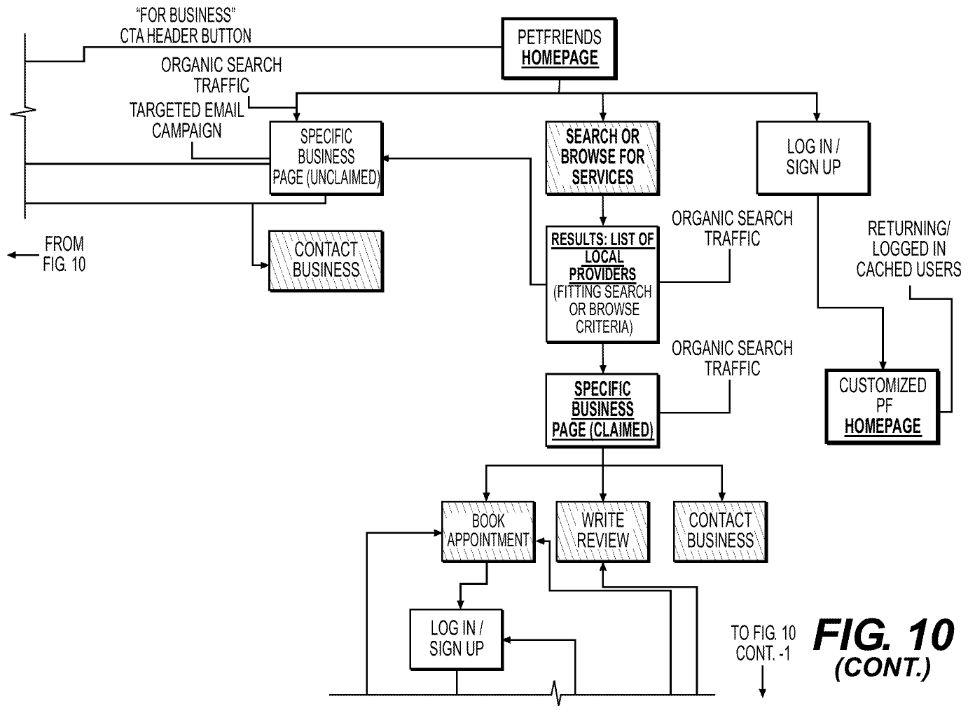

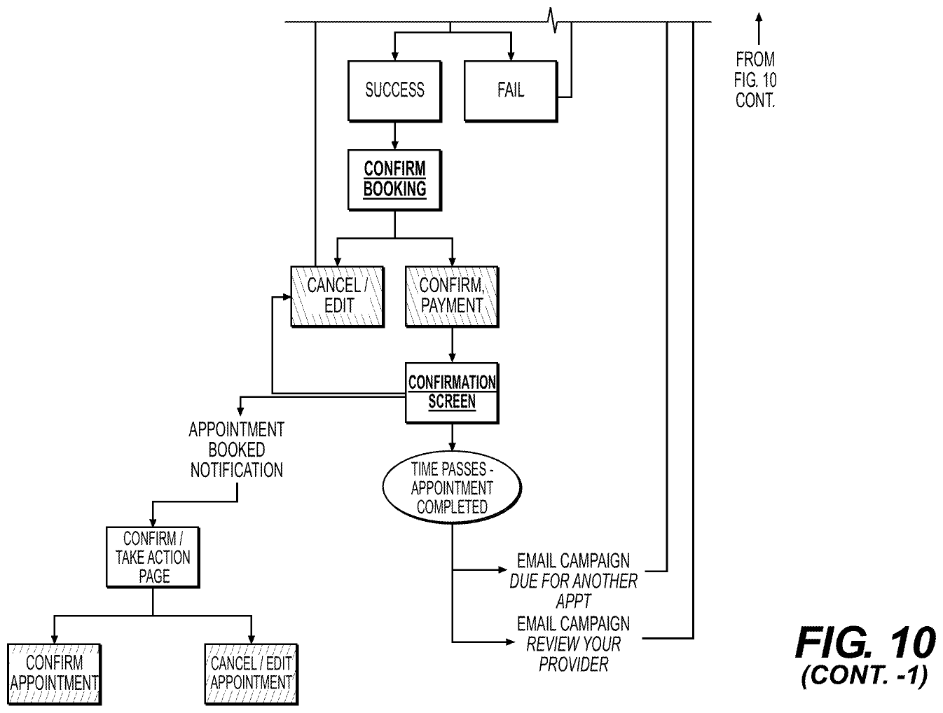

FIG. 10 describes an exemplary flowchart illustrating actions that may be performed by users of the web platform, according to one or more embodiments.

The web platform may allow providers to easily set future availability and capacity for appointments, including specific employee's availability, confirm/edit/cancel appointment requests, and/or maintain/update their business information and offerings. Feature functionality for providers may include a business profile/listing, a management dashboard, a custom report card, a notification center, and an interface for taking action corresponding to requests. The business profile/listing may include a digital storefront for providers. The management dashboard may be an interface where providers can update and/or change their business information displayed on their listing page. The custom report card may be a customizable post-service report that the provider may send to users after their pet's service. The notification center may be an interface that gives providers updates on requests, cancellations, etc. The interface for taking action corresponding to requests may allow providers to confirm, deny, or suggest an alternate time on appointment requests. Such an interface may give providers a clear picture of their upcoming schedule, as well as enable the providers to turn off their availability after accepting clients.

For example, as illustrated in FIG. 10, from the homepage, a service provider may select a “For Businesses” call-to-action (CTA) header button. The web platform may direct the service provider to a “For Businesses” landing page. If an unclaimed specific business page that corresponds to the service provider exists, the service provider of the corresponding specific business page may claim the business page. The web platform may direct the service provider to an “about” page for businesses. The web platform may authenticate the service provider by calling the service provider or texting the service provider. Authenticating the service provider allows for the web platform to validate/verify that the service provider does correspond to the specific business page. For example, the service provider may be able to verify their ownership of the business by using a code that will allow them to claim ownership of the business page via email, text, and/or phone call. If the service provider does not have the correct credentials to verify the identity, the web platform may inform the service provider to contact support. Alternatively, if the service provider does provide the correct credentials and has a positive verification, the web platform may allow the service provider to create a provider login/registration. Example fields of information that may be requested to register the provider's business may include a business type, a business phone number, a business website, a business address, hours of operation, business established date, and/or the provider's email. Alternatively, if no unclaimed business page exists, the service provider may submit to add the service provider to the web platform, then register the business page, and the web platform may allow the service provider to create a provider login/registration. Upon creating the provider login/registration, the service provider may be able to edit/update the service provider's profile page, set a schedule/availability/open appointments, as well as view/interact with the web platform's provider profile/dashboard. For example, the web platform may prompt the provider to upload business photos, include links to the business's social media profiles, include services menu and prices, add resources and/or employees for the business, and/or set a schedule/open available time slots (per employee or generally). The provider may also be provided with a dashboard, which may include a consolidated view of the provider's appointment requests, confirmed appointments, and the ability to access a customer list. From the dashboard, the provider may be able to edit or cancel a confirmed appointment, accept/confirm new appointment requests, and/or edit the provider's schedule, business information, etc. The appointment requests and confirmed appointments may display a limited summary of the details of the appointment, such as, for example, the services requested for the appointment. The provider may be able to check the details of the appointments by selecting the appointment that will bring up a view that has additional details.

From the homepage, a user may search for a particular service provider. If an unclaimed specific business page exists that corresponds to the particular service provider, the user may contact the particular service provider via the unclaimed specific business page.

From the homepage, a user may search or browse for pet care services. In response to the searching or browsing for services, the web platform may display a list of service providers. The user may select a claimed specific business page from the list of service providers. In response to selecting the claimed specific business page, the web platform may allow a user to book an appointment, write a review, and/or contact the business of the service provider. If the user selects the option to book an appointment, the web platform may prompt the user to log in/sign up to book the appointment. If the user fails to log in/sign up, the web platform may prompt the user to try to log in/sign up again. If the user successfully logs in/signs up, the user may proceed with booking the appointment, and the web platform may confirm the booking. Upon the web platform confirming the booking, the user may be able to cancel/edit the booking and/or confirm the booking by selecting a payment option. Upon confirming the booking by selecting a payment option, the web platform may present a confirmation screen. After time passes and the appointment is completed, the user may receive an email campaign reminding the user to review the service provider and/or remind the user that the pet is due for another appointment.

The web platform may send an appointment booked notification to a corresponding service provider. The web platform may provide the option for the service provider to confirm or cancel/edit the appointment.

### Exemplary User Profile Page

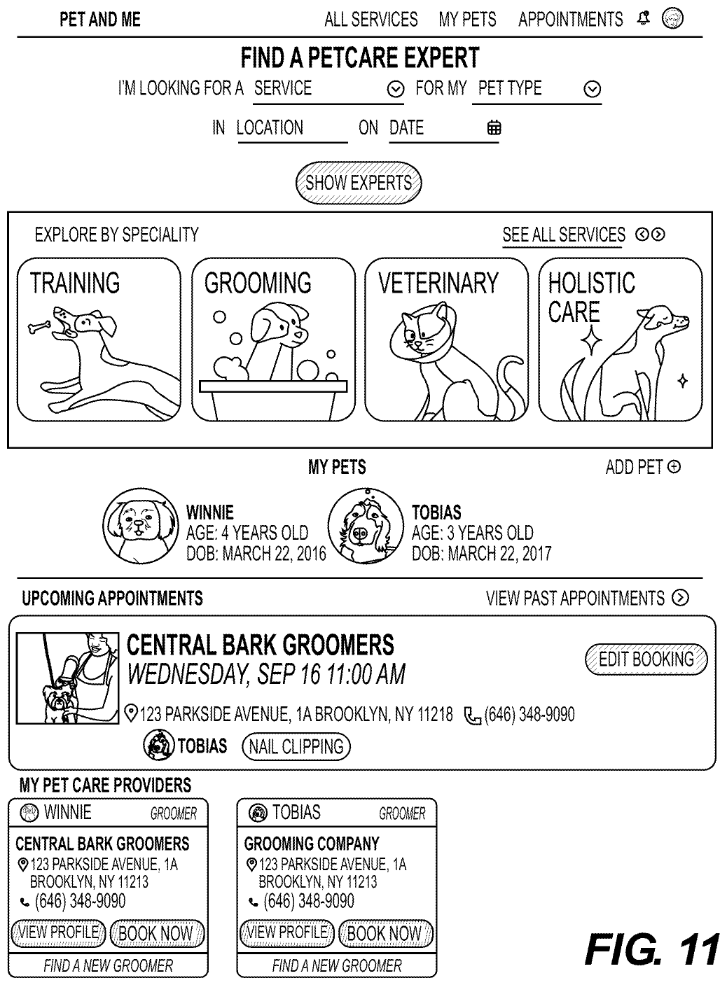

FIG. 11 illustrates an exemplary user profile page, according to one or more embodiments.

A user may engage with the web platform as a registered user (e.g., signed in to a user profile) or an unregistered user (e.g., does not have a user profile). If an unregistered user attempts to reserve services, schedule an appointment, and/or message a provider, such unregistered user may be directed to register/sign-up to the web platform before continuing further. Unregistered users may browse, search for services, browse business listings/search results, and/or view specific business pages. When unregistered users attempt to take action on a page (e.g., book services), they may be prompted to sign up or log in to the web platform. However, such unregistered users may be able to browse the site, business listings, and services offered, without creating a user profile first. Registration may be simple, where the requesting process may only require an email and password. The email may be required to be verified before booking services.

If a user is a registered user, a user profile page may be displayed if the user is logged in to the web platform. Registered, logged-in returning users of the web platform may have a user profile displaying the user's pets, upcoming appointments, past providers, and customized content. For example, the user profile may include a selection for services, pet type, location, date, and a specific business. The services and pet type selections may be natural language selections. The location selection may be an open ended search that may prompt a user for a zip code and/or an address. Auto-completion may be used, which may be based on a partial entry from a mapping API. An error may be returned if such selections are not filled and no specific business is searched. The date selection may pull up a calendar view with today and future dates highlighted and made available to be clicked. The specific business selection may be open ended, and such selection may utilize auto-completion based on a partial entry. The specific business selection may be an optional type of search. Additionally, for example, the customized content may include a content hub, which may include provider blogs, articles, and the like. The content hub may also provide one or more leads.

A pet profile banner may also be displayed to the user. The pet profile banner may include one or more pet profiles that are associated with the user profile. In some embodiments, the pet profile banner may include the pet(s) profile images of the one or more pet profiles. The pet profile banner may also include the name, age, and/or date of latest past service(s) booked/completed. Such pet profile information may be displayed for each of the one or more pet profiles.

An upcoming appointments notification section may also be displayed to the user. If there are upcoming appointments for one or more pets, such appointments may be displayed in descending order from soonest to latest appointments scheduled. Information to be displayed with the appointment may include which pet the appointment is booked for, the provider/business servicing the appointment, the provider address, the specific resource booked, the day and time of the appointment, the specific services booked for the appointment, and/or an option to edit the booking. A section displaying recently used or bookmarked providers may also be displayed to the user. Notifications and/or reminders may include automated emails and/or texts to remind users of upcoming appointments. An “add appointment to calendar” option may provide the ability for users to add booked appointments to their electronic calendar.

### Exemplary Pet Profile

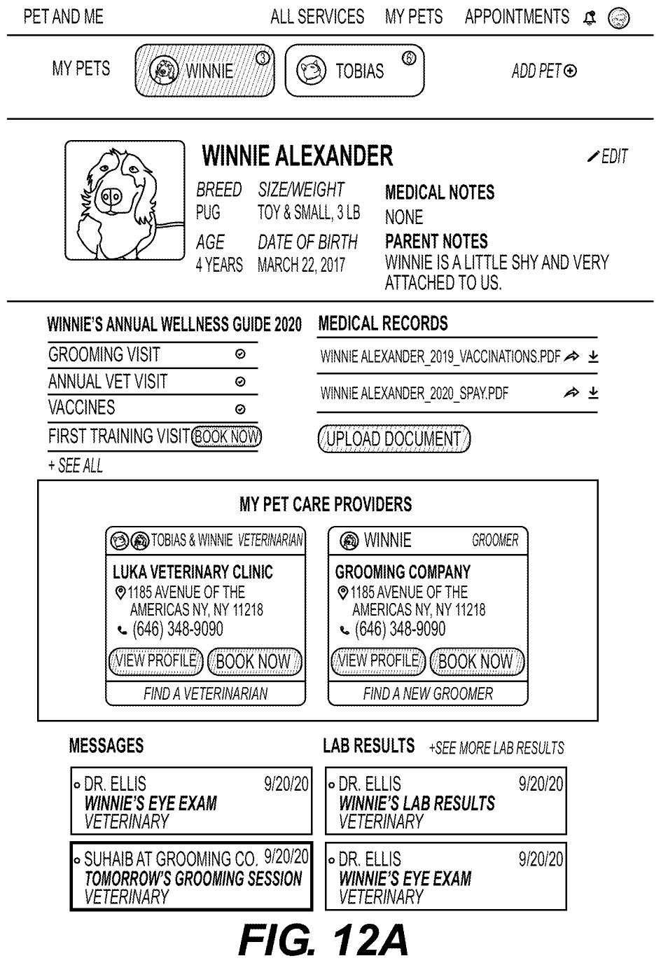

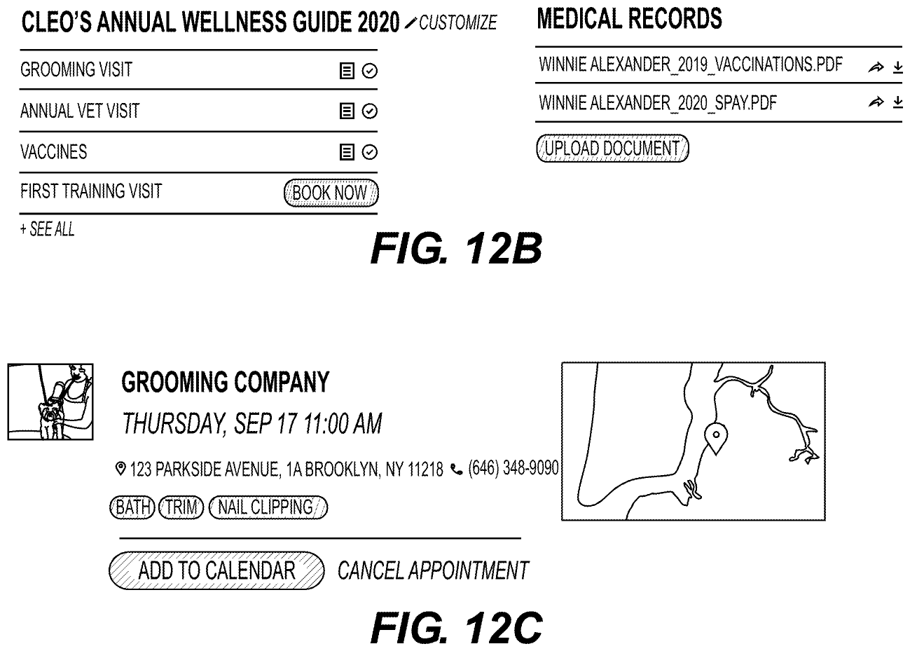

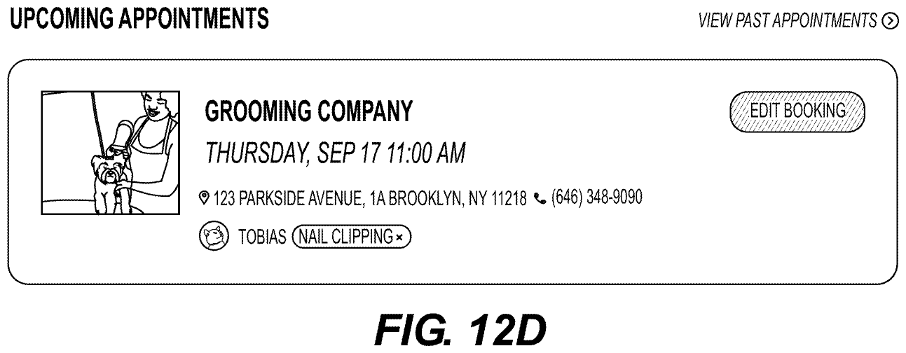

FIGS. 12A-D and 13 illustrate exemplary pet profile pages, according to one or more embodiments.

Each pet may have a corresponding pet profile, where the pet profile may be associated with one or more user profiles. The pet profile may include information corresponding to the pet, such as the pet's name, breed, size, birthdate, medical notes, and/or parent notes. The pet profile may also include a wellness guide, upcoming appointments, pet care providers, messages, lab results, and/or medical records. The wellness guide may include particular service provider visit thresholds (e.g., a grooming visit, an annual vet visit, a number of vaccines, a training visit, and the like) for the pet to meet during a particular time frame (e.g., a year). The upcoming appointments may correspond to any bookings that are upcoming for the pet. The pet care providers may include one or more service providers that have performed services for the pet in the past. Additionally, or alternatively, the pet care providers may include providers that have been designated as “preferred” by the web platform and/or the user. The messages may include one or more messages from service providers, other users, and the like. The lab results may be automatically uploaded from the pet's veterinarian (e.g., an external third party system). In some embodiments, the pet's veterinarian may be aware of the pet profile and may automatically upload the pet's lab results, medical records, and the like.

The medical records may include medical records received from one or more veterinarians. There may be at least two ways that the pet profile may receive the pet's medical records. First, there may be a medical records upload form in the pet profile page, where a user may upload their pet's records. Feature functionality may include the ability to upload veterinary records, which may provide users with the ability to upload files of the user's pet's medical/vaccination records, where the pet's medical/vaccine records may be sent to providers upon request.

Second, a “Vet Record Retrieval” option may exist for users. Feature functionality may include uploading veterinary records and/or requesting veterinary records. Requesting veterinary records functionality may provide users with the ability to select their pet's veterinarian and request the retrieval of the medical/vaccination records from the veterinarian. For example, customers may request their pet's medical records through the pet profile. The request may include a digital consent form that may be used to retrieve the pet vaccination records from the pet's veterinarian. When a user submits a “Vet Record Retrieval” request, an email (or fax) may automatically be sent to the pet's veterinary service provider with the request and the consent. Upon receiving the vaccination records from the pet's veterinary service provider, a management dashboard may upload the records to the customer's profile. In some embodiments, the medical records may be provided without any explicit request from a user. For example, the medial records may be provided to the web platform (or a component of the web platform that management pet profiles) upon the veterinary service provider's request to add the medical records to the pet's profile, or upon the medical records being recorded by the veterinary service provider using a computing device. The receipt of the medical records may trigger a notification that is sent to the customer, where the notification indicates that the pet's medical records have been successfully (or unsuccessfully) retrieved from the veterinarian. Once the pet's medical records have been uploaded, the user may be able to download and/or email themselves (or other people) the medical records. In some embodiments, the web platform may automatically send the pet's medical records to one or more external third party pet services (e.g., groomers, trainers, and the like).

In some embodiments, at the time of booking a service, providers may need to have a pet's vaccine/medical records on file. For example, some providers may have particular vaccine/medical record requirements that the pet needs to meet in order to confirm the booking. Existing customers who have pets with up-to-date vaccinations may already have the vaccine records on file. However, new customers, or returning customers with lapsed records, may not have current vaccine records on file. A service provider, via the web platform, may send a request to the component of the web platform that manages the pet profile (e.g., a pet profile component) for the pet's vaccine/medical records. In some embodiments, the service provider, via the web platform, may send a request to the pet profile component to see if the pet's vaccine/medical records meet a requirement of the service provider. In such an embodiment, the pet profile component's response may include “yes,” “no,” or “undetermined.” Such an embodiment may preserve the pet's medical data by not revealing the pet's specific medical data to the service provider. Additionally, the service provider may deny the booking if the pet does not meet the medical requirements of the service provider.

In some embodiments, after completing sign up or log in, a user may proceed with a particular booking and add additional information (e.g., pet details) to the user's profile in order to complete the booking. For example, if a user does not have a pet added to the user's profile, the user may be prompted to add the pet. The web platform may request pet information, such as a pet name, a pet birthday, and/or breed information. The user may then select an open time window with the provider and apply a pet to the appointment request. If the user has more than one pet and is seeking the same service (e.g., grooming) for all of the pets, the web platform may default to display providers that have indicated that they can accept more than one pet. Upon following the booking steps, there may be a simple indicator that the appointment has been requested and is pending confirmation from the provider. Once the appointment has been confirmed or denied, the web platform may send an email indicating the confirmation or denial of the appointment.

### Exemplary Method for Managing Electronic Transactions of Pet Care Services Data

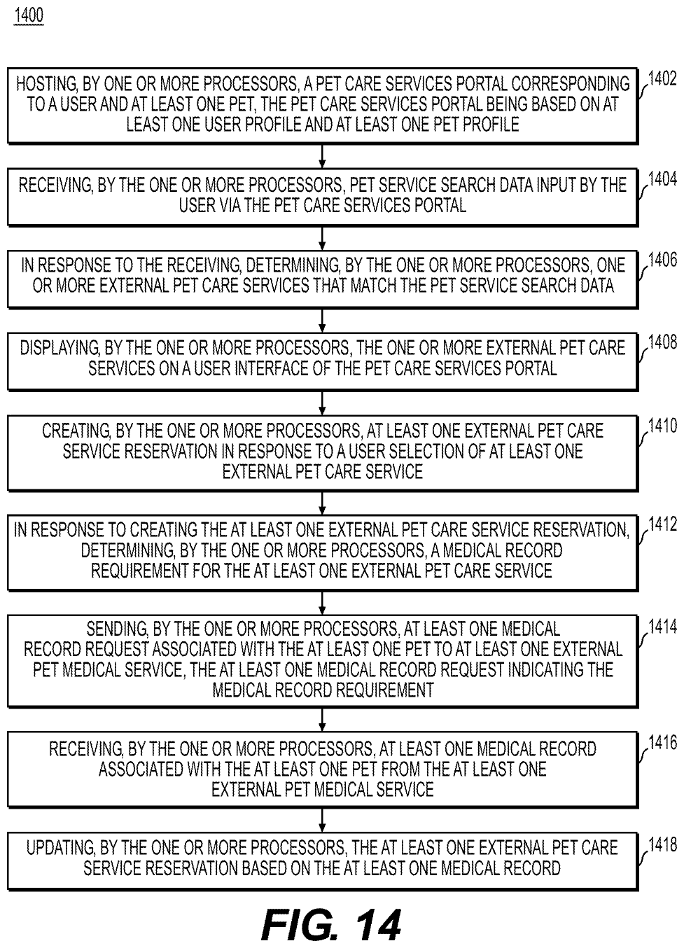

FIG. 14 illustrates an exemplary method 1400 of an exemplary embodiment for dynamically managing electronic transactions of pet care services data, according to one or more embodiments. Notably, method 1400 may be performed by one or more processors of a server that is in communication with one or more user devices and other external system(s) via a network. However, it should be noted that method 1400 may be performed by any one or more of the server, one or more user devices, or other external systems.

The method may include hosting, by one or more processors, a pet care services portal corresponding to a user and at least one pet, the pet care services portal being based on at least one user profile and at least one pet profile (Step 1402). For example, the pet care services portal may be hosted by the web platform. The pet care services portal may include the booking engine. Additionally, the pet care services portal may be customized based on information from the at least one user profile and/or the at least one pet profile.

The method may also include receiving, by the one or more processors, pet service search data input by the user via the pet care services portal (Step 1404). The pet service search data may include a service, a pet type, a location, and/or a date. The service may include one or more of: training, grooming, veterinary, and/or holistic care. The location may be input by the user or automatically determined by the web platform. The date may include the current day and future days for a period of days, weeks, or years.

The method may also include, in response to the receiving, determining, by the one or more processors, one or more external pet care services that match the pet service search data (Step 1406). The one or more external pet care services may include at least one training pet care service, at least one grooming pet care service, at least one veterinary pet care service, or at least one holistic pet care service. The one or more external pet care services may include at least one attribute that matches at least part of the pet service search data.

The method may also include displaying, by the one or more processors, the one or more external pet care services on a user interface of the pet care services portal (Step 1408). In some embodiments, additional details corresponding to the one or more external pet care service may also be displayed on the user interface. The additional details may include one or more ratings, a distance, a time period of the existence of the external pet care service, one or more certifications, one or more accreditations, a picture, and/or one or more available times/dates for reserving a service.

The method may also include creating, by the one or more processors, at least one external pet care service reservation in response to a user selection of at least one external pet care service (Step 1410). In some embodiments, creating the at least one external pet care service reservation may include associating the at least one external pet care service reservation with the at least one external pet care service. The associating may include creating at least one database record that includes information corresponding to the at least one external pet care service reservation. The at least one database record may also include a unique identifier associated with the at least one external pet care service. In some embodiments, the associating may include the web platform sending a notification to the at least one external pet care service, where the notification includes the details of the at least one pet care service reservation. The notification may allow for the at least one pet care service to confirm, edit, and/or deny the at least one external pet care service reservation. In some embodiments, if the at least one pet care service confirms the at least one external pet care service reservation, the user may receive a confirmation notification. If the at least one pet care service edits the at least one external pet care service reservation, the user may receive a notification of the modification. If the at least one pet care service denies the at least one external pet care service reservation, the user may receive a notification of the denial.

Additionally, the method may also include associating, by the one or more processors, the at least one external pet care service reservation with the at least one user profile or the at least one pet profile. The associating may include creating at least one database record that includes information corresponding to the at least one external pet care service reservation. The at least one database record may also include a unique identifier associated with the at least one user profile and/or the at least one pet profile. In some embodiments, the associating may include the web platform sending a notification to the at least one user profile or the at least one pet profile, where the notification includes the details of the at least one pet care service reservation. The notification may allow for the at least one user profile or the at least one pet profile to confirm, edit, and/or deny the at least one external pet care service reservation.

The method may also include, in response to creating the at least one external pet care service reservation, determining, by the one or more processors, a medical record requirement for the at least one external pet care service (Step 1412). The medical record requirement may correspond to particular vaccination requirements and/or other medical requirements of the at least one external pet care service. For example, the medical record requirement may include a vaccination requirement that a pet has received a rabies vaccination and a distemper vaccination. Additionally, in some embodiments, the medical record requirement may also include a time threshold. For example, the medical record requirement may include that the pet has had a rabies vaccination and a distemper vaccination in the past year. In some embodiments, the other medical requirements may include whether the pet has been neutered/spayed.

The method may also include sending, by the one or more processors, at least one medical record request associated with the at least one pet to at least one external pet medical service, the at least one medical record request indicating the medical record requirement (Step 1414). The medical record request may be sent to at least one external pet medical service, such as a veterinarian, clinic, urgent care, and the like. The medical record request may include some or all of the medical record requirement. For example, a medical record request may be sent to a veterinarian associated with the pet, where the medical record request includes a requirement that the pet had a rabies vaccination in the past year.

In some embodiments, the sending the at least one medical record request associated with the at least one pet to the at least one external pet medical service may include determining, by the one or more processors, at least one pet identifier and the at least one external pet medical service based on the at least one pet profile, wherein the at least one medical record request includes the at least one pet identifier. The pet identifier may include a pet microchip identifier, where the at least one medical record request may include the pet microchip identifier. The method may include analyzing the at least one pet profile to determine at least one pet identifier, such as a pet microchip identifier. Additionally, the method may include analyzing the at least one pet profile to determine at least one external pet medical service that is associated with the at least one pet profile. For example, the at least one pet profile may be associated with a veterinarian. Additionally, in some embodiments, the determining at least one external pet medical service may include sending a confirmation request to a plurality of default external pet medical services. The confirmation request may include the at least one pet identifier, where the confirmation request may ask each of the plurality of default external pet medical services to confirm whether they have at least one medical/vaccination record corresponding to the at least one pet identifier. For example, the plurality of default external pet medical services may include some or all of the veterinarian services that are registered with the web platform. The plurality of default external pet medical services may correspond to pet medical services that are not associated with the at least one pet identifier. If the web platform receives a confirmation that a default external pet medical service has at least one medical/vaccination record corresponding to the at least one pet identifier, the web platform may store the at least one medical/vaccination record and associate the at least one medical/vaccination record with the at least one pet profile. Additionally, the web platform may also associate the at least one pet profile with at least one of the default external pet medical service.

The method may also include receiving, by the one or more processors, at least one medical record associated with the at least one pet from the at least one external pet medical service (Step 1416). For example, the at least one medical record may include a portion or all of the pet's medical record. In some embodiments, the at least one medical record may include an indicator regarding whether the pet's medical record fulfills the medical record requirement, without revealing the pet's medical/vaccination details. For example, the at least one external pet medical service may analyze the pet's internal medical records to determine whether the pet satisfies the medical record requirement. The indicator may include “yes,” “no,” or “undetermined.”

The method may also include updating, by the one or more processors, the at least one external pet care service reservation based on the at least one medical record (Step 1418). The updating may include adding an indicator regarding whether the pet satisfies the medical record requirement. The indicator may include “yes,” “no,” or “undetermined.” Additionally, or alternatively, the updating may include populating the at least one external pet care service reservation with some or all of the information from the at least one medical record. For example, the at least one external pet care service reservation may be populated to include one or more vaccinations, one or more vaccination dates, and/or additional medical information from the at least one medical record.

In some embodiments, the method may also include determining, by the one or more processors, the grooming history or training history for the at least one external pet care service. Additionally, in some embodiments, the determining the grooming history or the training history may include sending a confirmation request to a plurality of default external pet grooming services and/or a plurality of default external pet training services. For example, the plurality of default external pet grooming services and/or the plurality of default external pet training services may include some or all of the grooming/training services that are registered with the web platform. The confirmation request may include the at least one pet identifier, pet name, and/or other identifying information, where the confirmation request asks each of the plurality of default external pet grooming services and/or a plurality of default external pet training services to confirm whether the pet has engaged in the grooming services and/or training services. If the web platform receives a confirmation that a default external pet grooming service and/or a default external pet training service has at least one grooming/training record corresponding to the at least one pet identifier, the web platform may store the at least one grooming/training record and associate the at least one grooming/training record with the at least one pet profile. Additionally, the web platform may also associate the at least one pet profile with the default external pet grooming service and/or the default external pet training service.

The method may also include associating, by the one or more processors, the grooming history or the training history with the at least one medical record. The associating may include creating (or updating) at least one database record that includes information corresponding to the at least one pet profile, the grooming and/or the training history, and/or the at least one medical record. The at least one database record may also include a unique identifier associated with the at least one user profile and/or the at least one pet profile. In some embodiments, all or a portion of the information stored in relation to the pet (e.g., medical records, grooming history, training history, holistic care history, etc.) may be accessible by one or more external pet care services associated with the pet. For example, as discussed elsewhere in the current disclosure, medical records received from a veterinary service provider may be provided to one or more other external pet care services. As another example, the grooming history, training history, and/or holistic care history may be accessible by a veterinary service provider, in order for the service provider to make a more informed decision about the pet's medical care. Therefore, various types of information stored at the web platform in relation to a pet may be shared among multiple external pet care services, in accordance with certain rules or restrictions (e.g., access rules or restrictions) defined for each external pet care service. In some embodiments, the associating may include the web platform sending a notification to the at least one user profile or the at least one pet profile, where the notification includes the details of the grooming history and/or the training history. The notification may allow for the at least one user profile or the at least one pet profile to confirm, edit, and/or deny the grooming history and/or the training history.

In some embodiments, wherein the one or more external pet care services include at least one grooming pet care service, the method may further include displaying, by the one or more processors, a plurality of recommended grooming style images. For example, the recommended grooming style images may correspond to the breed, hair length, size, and other attributes of the pet. The recommended grooming style images may include images of potential grooming styles for the pet. The plurality of grooming style images may be displayed on a user interface, such as a user interface of a user device. The method may further include receiving, by the one or more processors, at least one grooming style user selection from the plurality of recommended grooming style images. The user may select at least one grooming style of the displayed plurality of recommended grooming styles. For example, there may be a particular grooming style that the user would like the groomer to emulate when grooming the user's pet. In some embodiments, the at least one grooming style user selection may be saved for future reference.

Although FIG. 14 shows example blocks of exemplary method 1400, in some implementations, the exemplary method 1400 may include additional blocks, fewer blocks, different blocks, or differently arranged blocks than those depicted in FIG. 14. Additionally, or alternatively, two or more of the blocks of the exemplary method 1400 may be performed in parallel.

### Exemplary Environment and Exemplary Device

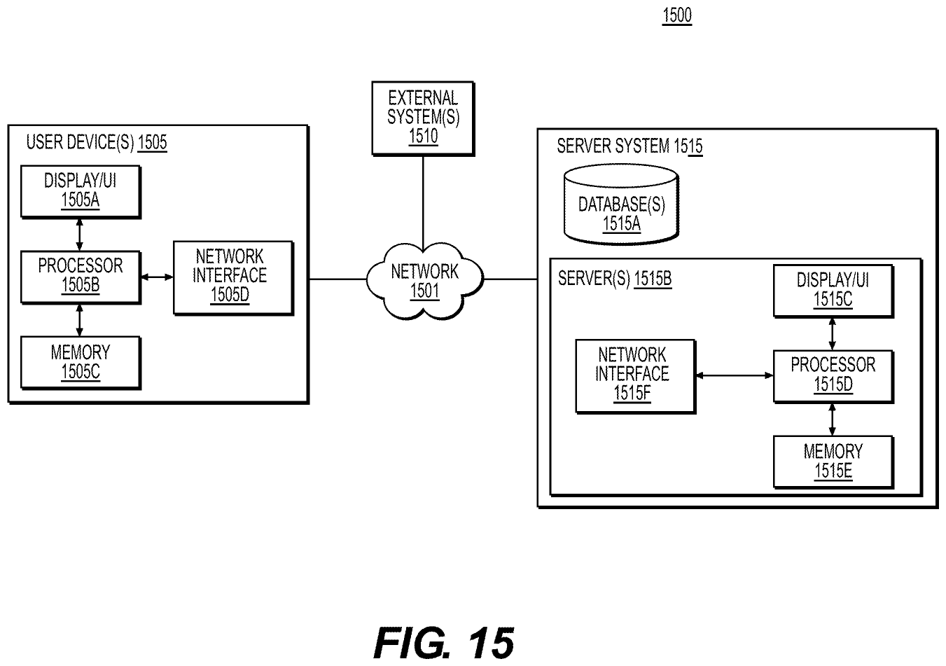

FIG. 15 depicts an exemplary environment 1500 that may be utilized with techniques presented herein. One or more user device(s) 1505, one or more external system(s) 1510, and one or more server system(s) 1515 may communicate across a network 1501. As will be discussed in further detail below, one or more server system(s) 1515 may communicate with one or more of the other components of the environment 1500 across network 1501. The one or more user device(s) 1505 may be associated with a user, e.g., a user associated with dynamically managing electronic transactions of pet care services data.

In some embodiments, the components of the environment 1500 are associated with a common entity, e.g., a veterinarian, clinic, animal specialist, research center, or the like. In some embodiments, one or more of the components of the environment is associated with a different entity than another. The systems and devices of the environment 1500 may communicate in any arrangement. As will be discussed herein, systems and/or devices of the environment 1500 may communicate in order to dynamically manage electronic transaction of pet care services data, among other activities.

The user device 1505 may be configured to enable the user to access and/or interact with other systems in the environment 1500. For example, the user device 1505 may be a computer system such as, for example, a desktop computer, a mobile device, a tablet, etc. In some embodiments, the user device 1505 may include one or more electronic application(s), e.g., a program, plugin, browser extension, etc., installed on a memory of the user device 1505.

The user device 1505 may include a display/user interface (UI) 1505A, a processor 15058, a memory 1505C, and/or a network interface 1505D. The user device 1505 may execute, by the processor 1505B, an operating system (O/S) and at least one electronic application (each stored in memory 1505C). The electronic application may be a desktop program, a browser program, a web client, or a mobile application program (which may also be a browser program in a mobile O/S), an applicant specific program, system control software, system monitoring software, software development tools, or the like. For example, environment 1500 may extend information on a web client that may be accessed through a web browser. In some embodiments, the electronic application(s) may be associated with one or more of the other components in the environment 1500. The application may manage the memory 1505C, such as a database, to transmit streaming data to network 1501. The display/UI 1505A may be a touch screen or a display with other input systems (e.g., mouse, keyboard, etc.) so that the user(s) may interact with the application and/or the O/S. The network interface 1505D may be a TCP/IP network interface for, e.g., Ethernet or wireless communications with the network 1501. The processor 15058, while executing the application, may generate data and/or receive user inputs from the display/UI 1505A and/or receive/transmit messages to the server system 1515, and may further perform one or more operations prior to providing an output to the network 1501.

External systems 1510 may be, for example, one or more third party and/or auxiliary systems that integrate and/or communicate with the server system 1515 in performing various document information extraction tasks. External systems 1510 may be in communication with other device(s) or system(s) in the environment 1500 over the one or more networks 1501. For example, external systems 1510 may communicate with the server system 1515 via API (application programming interface) access over the one or more networks 1501, and also communicate with the user device(s) 1505 via web browser access over the one or more networks 1501.

In various embodiments, the network 1501 may be a wide area network (“WAN”), a local area network (“LAN”), a personal area network (“PAN”), or the like. In some embodiments, network 1501 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 1515 may include an electronic data system, e.g., a computer-readable memory such as a hard drive, flash drive, disk, etc. In some embodiments, the server system 1515 includes and/or interacts with an application programming interface for exchanging data to other systems, e.g., one or more of the other components of the environment.

The server system 1515 may include a database 1515A and at least one server 1515B. The server system 1515 may be a computer, system of computers (e.g., rack server(s)), and/or or a cloud service computer system. The server system may store or have access to database 1515A (e.g., hosted on a third party server or in memory 1515E). The server(s) may include a display/UI 1515C, a processor 1515D, a memory 1515E, and/or a network interface 1515F. The display/UI 1515C may be a touch screen or a display with other input systems (e.g., mouse, keyboard, etc.) for an operator of the server 15158 to control the functions of the server 15158. The server system 1515 may execute, by the processor 1515D, an operating system (O/S) and at least one instance of a servlet program (each stored in memory 1515E).

Although depicted as separate components in FIG. 15, it should be understood that a component or portion of a component in the environment 1500 may, in some embodiments, be integrated with or incorporated into one or more other components. For example, a portion of the display 1515C may be integrated into the user device 1505 or the like. In some embodiments, operations or aspects of one or more of the components discussed above may be distributed amongst one or more other components. Any suitable arrangement and/or integration of the various systems and devices of the environment 1500 may be used.

In the following methods, various acts may be described as performed or executed by a component from FIG. 15, such as the server system 1515, the user device 1505, or components thereof. However, it should be understood that in various embodiments, various components of the environment 1500 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

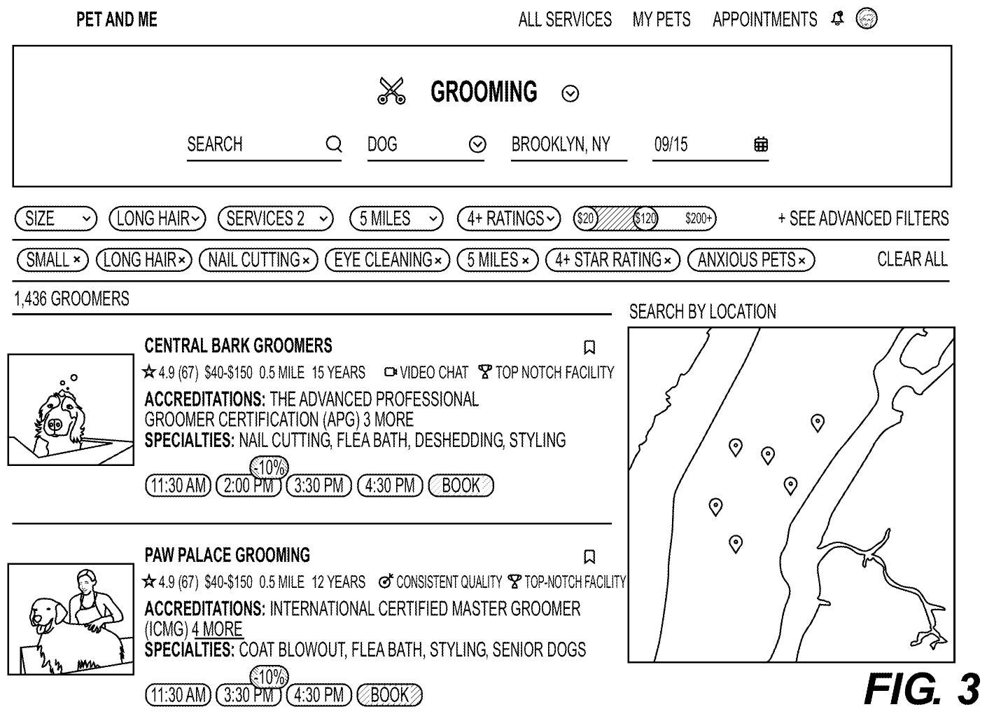

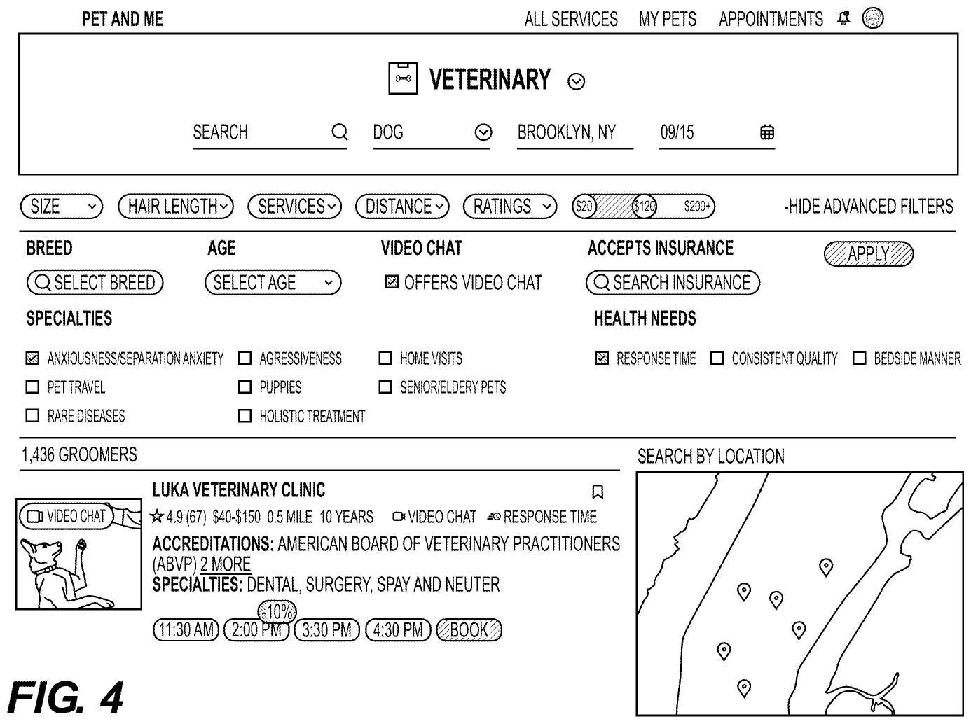

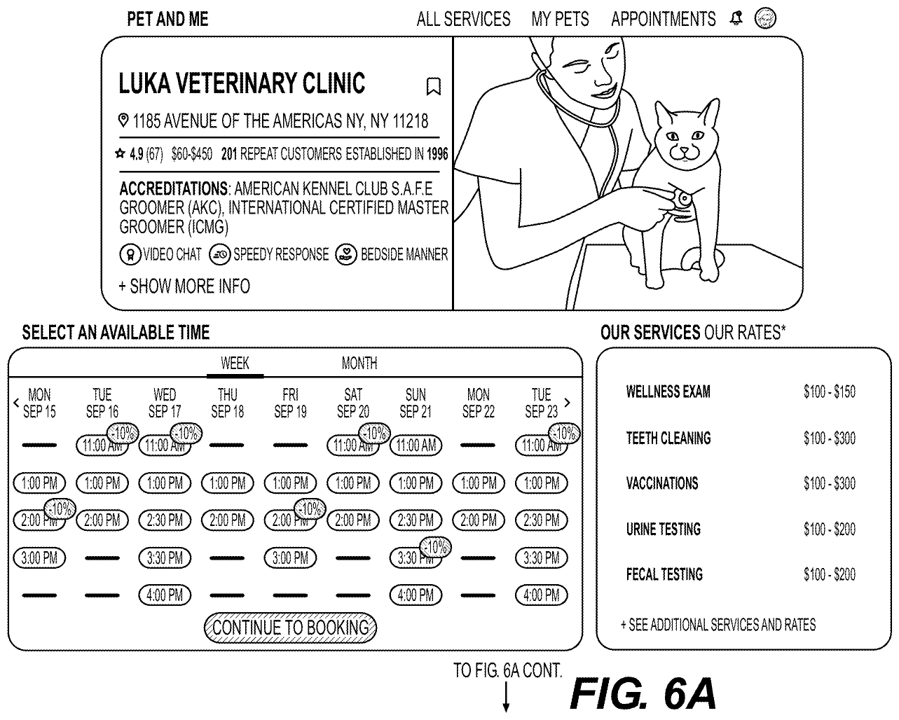

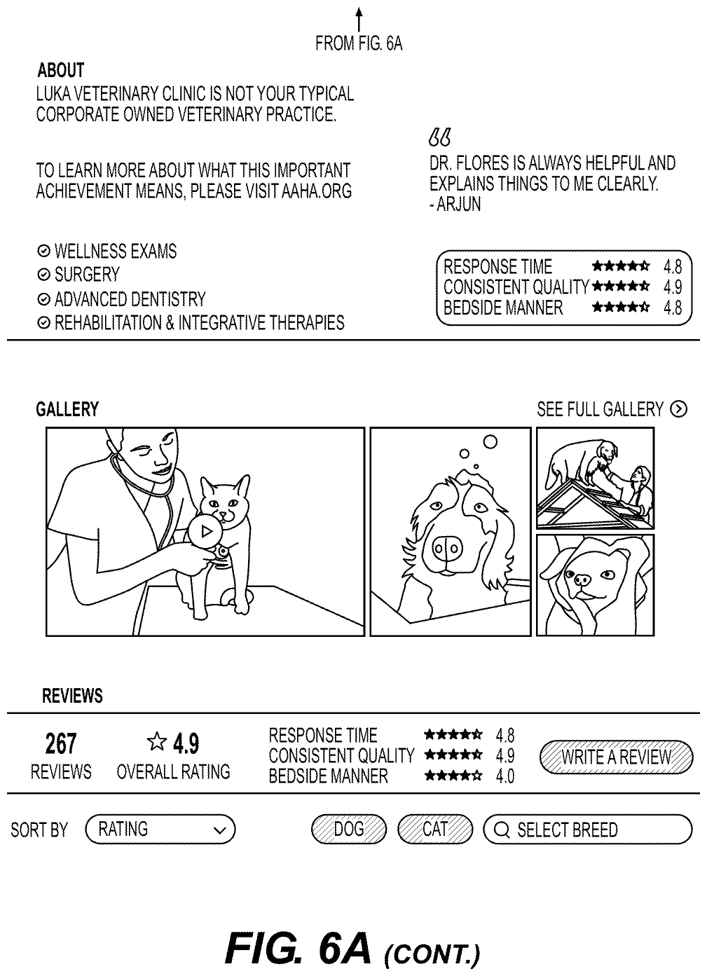

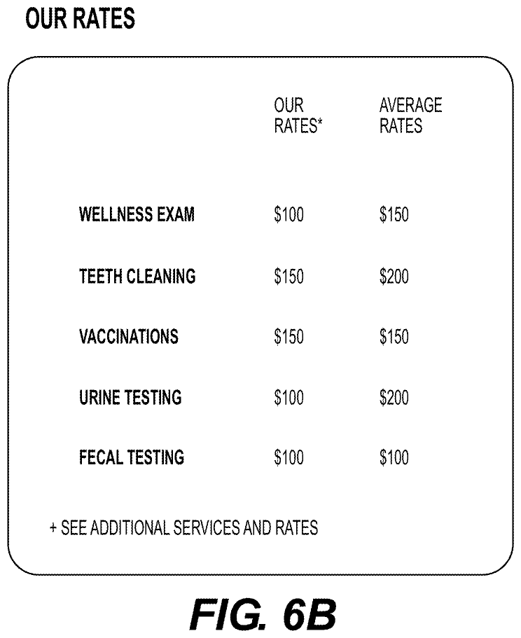

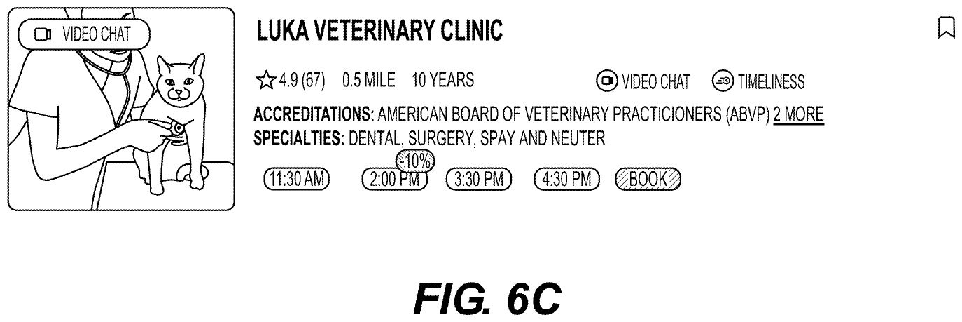

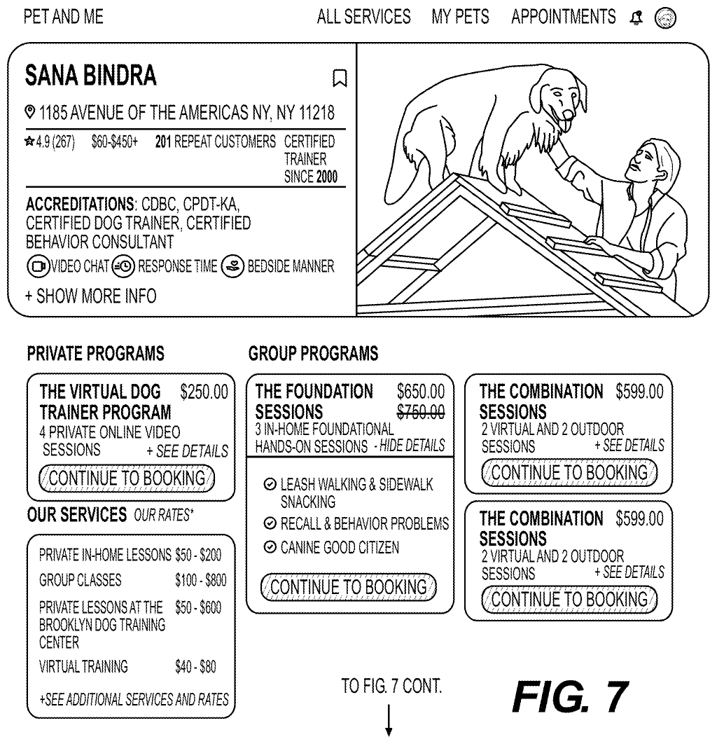

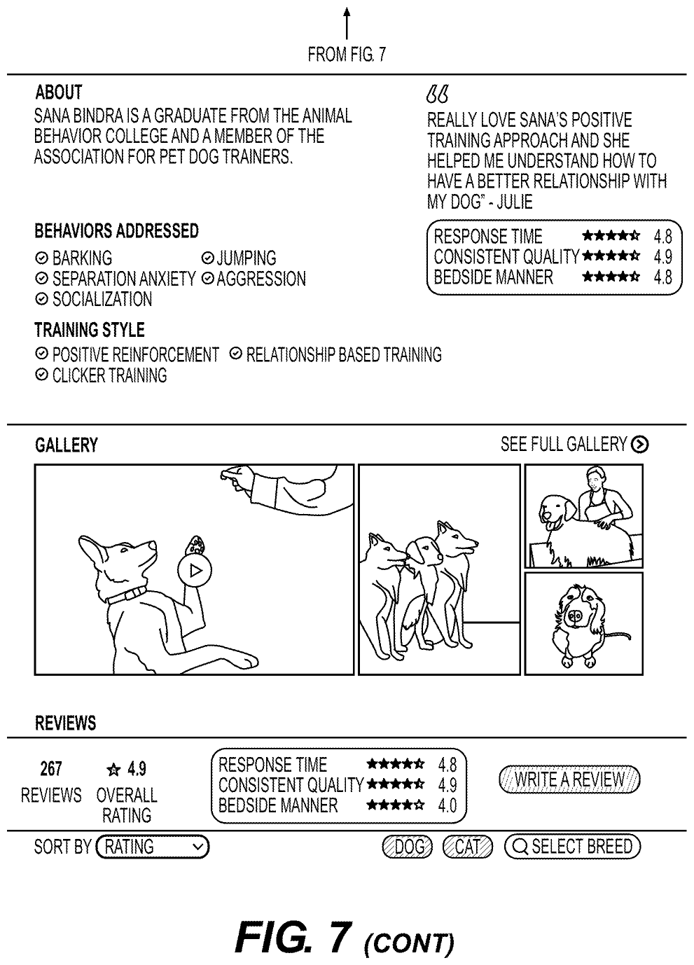

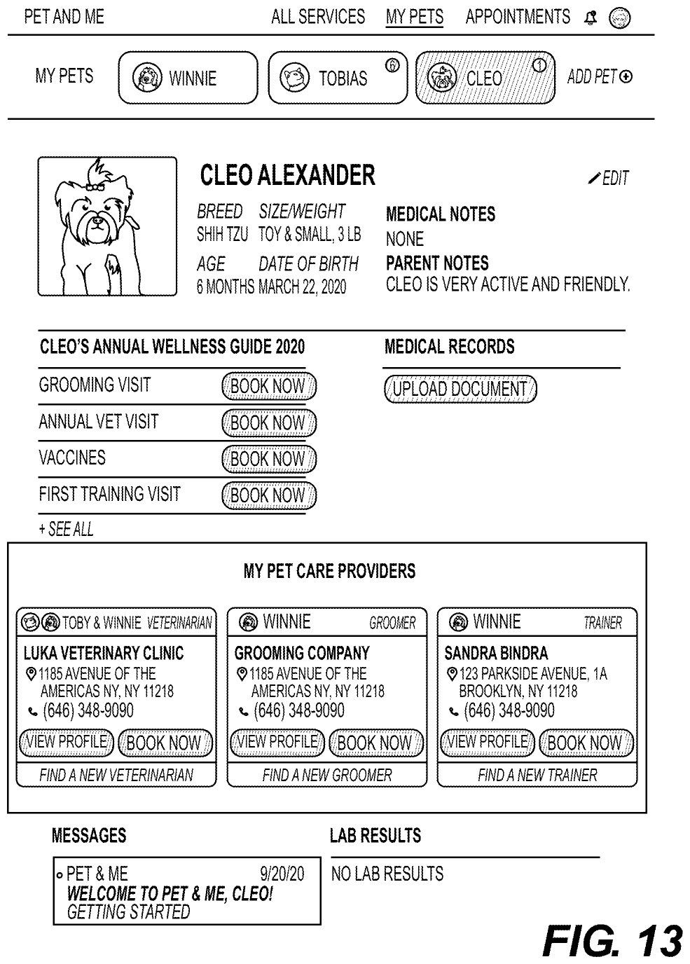

In general, any process or operation discussed in this disclosure that is understood to be computer-implementable, such as the processes illustrated in FIGS. 1A-14, may be performed by one or more processors of a computer system, such any of the systems or devices in the environment 1500 of FIG. 15, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 15. One or more processors of a computer system may be included in a single computing device or distributed among a plurality of computing devices. A memory of the computer system may include the respective memory of each computing device of the plurality of computing devices.

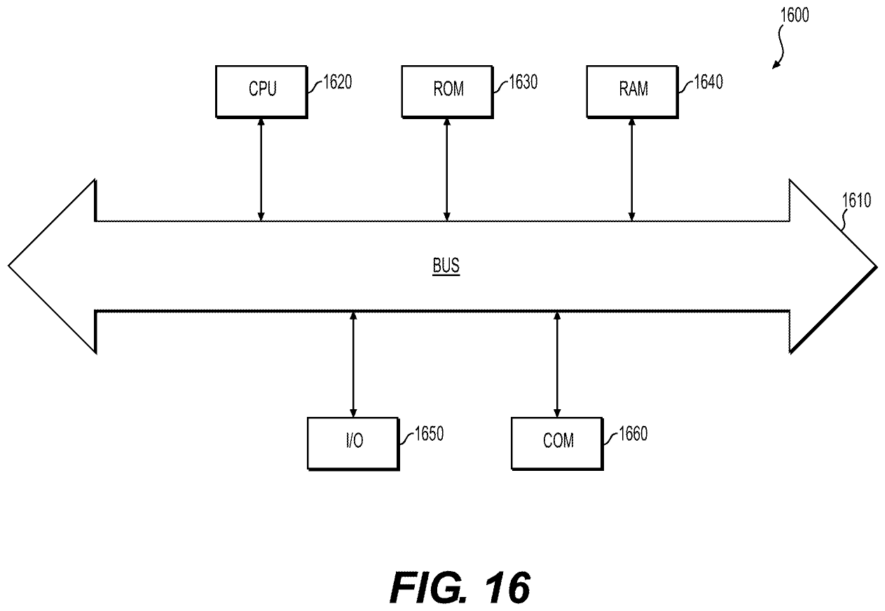

FIG. 16 is a simplified functional block diagram of a computer 1600 that may be configured as a device for executing the methods of FIGS. 1A-14, according to exemplary embodiments of the present disclosure. For example, device 1600 may include a central processing unit (CPU) 1620. CPU 1620 may be any type of processor device including, for example, any type of special purpose or a general-purpose microprocessor device. As will be appreciated by persons skilled in the relevant art, CPU 1620 also may be a single processor in a multi-core/multiprocessor system, such system operating alone, or in a cluster of computing devices operating in a cluster or server farm. CPU 1620 may be connected to a data communication infrastructure 1610, for example, a bus, message queue, network, or multi-core message-passing scheme.

Device 1600 also may include a main memory 1640, for example, random access memory (RAM), and also may include a secondary memory 1630. Secondary memory 1630, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 1630 may include other similar means for allowing computer programs or other instructions to be loaded into device 1600. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 1600.

Device 1600 also may include a communications interface (“COM”) 1660. Communications interface 1660 allows software and data to be transferred between device 1600 and external devices. Communications interface 1660 may include a modem, a network interface (such as an Ethernet card), a communications port, a PCMCIA slot and card, or the like. Software and data transferred via communications interface 1660 may be in the form of signals, which may be electronic, electromagnetic, optical, or other signals capable of being received by communications interface 1660. These signals may be provided to communications interface 1660 via a communications path of device 1600, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 1600 also may include input and output ports 1650 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the general description and the detailed description are exemplary and explanatory only and are not restrictive of the features, as claimed.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for dynamically managing electronic transactions of pet care services data, the method comprising:
hosting, by one or more processors, a pet care services portal corresponding to a user and at least one pet, the pet care services portal being based on at least one user profile and at least one pet profile;
receiving, by the one or more processors, pet service search data input by the user via the pet care services portal;
in response to the receiving, determining, by the one or more processors, one or more external pet care services that match the pet service search data;
displaying, by the one or more processors, the one or more external pet care services on a user interface of the pet care services portal;
creating, by the one or more processors, at least one external pet care service reservation in response to a user selection of at least one external pet care service;
in response to creating the at least one external pet care service reservation, determining, by the one or more processors, a medical record requirement for the at least one external pet care service;
sending, by the one or more processors, at least one medical record request associated with the at least one pet to at least one external pet medical service, the at least one medical record request indicating the medical record requirement;
receiving, by the one or more processors, at least one medical record associated with the at least one pet from the at least one external pet medical service; and
updating, by the one or more processors, the at least one external pet care service reservation based on the at least one medical record.

2. The computer-implemented method of claim 1, the method further comprising:
associating, by the one or more processors, the at least one external pet care service reservation with the at least one user profile or the at least one pet profile.

3. The computer-implemented method of claim 1, wherein creating the at least one external pet care service reservation includes associating the at least one external pet care service reservation with the at least one external pet care service.

4. The computer-implemented method of claim 1, the sending the at least one medical record request associated with the at least one pet to the at least one external pet medical service including:
determining, by the one or more processors, at least one pet identifier and the at least one external pet medical service based on the at least one pet profile, wherein the at least one medical record request includes the at least one pet identifier.

5. The computer-implemented method of claim 4, wherein the at least one pet identifier includes a pet microchip identifier.

6. The computer-implemented method of claim 1, wherein the one or more external pet care services include at least one training pet care service, at least one grooming pet care service, at least one veterinary pet care service, or at least one holistic pet care service.

7. The computer-implemented method of claim 1, wherein the one or more external pet care services include at least one grooming pet care service, and the method further comprises:
displaying, by the one or more processors, a plurality of recommended grooming style images; and
receiving, by the one or more processors, at least one grooming style user selection from the plurality of recommended grooming style images.

8. The computer-implemented method of claim 1, the method further comprising:
determining, by the one or more processors, grooming history or training history for the at least one external pet care service; and
associating, by the one or more processors, the grooming history or the training history with the at least one medical record.

9. A computer system for dynamically managing electronic transactions of pet care services data, the computer system comprising:
at least one memory storing instructions; and
at least one processor configured to execute the instructions to perform operations comprising:
hosting a pet care services portal corresponding to a user and at least one pet, the pet care services portal being based on at least one user profile and at least one pet profile;
receiving pet service search data input by the user via the pet care services portal;
in response to the receiving, determining one or more external pet care services that match the pet service search data;
displaying the one or more external pet care services on a user interface of the pet care services portal;
creating at least one external pet care service reservation in response to a user selection of at least one external pet care service;
in response to creating the at least one external pet care service reservation, determining a medical record requirement for the at least one external pet care service;
sending at least one medical record request associated with the at least one pet to at least one external pet medical service, the at least one medical record request indicating the medical record requirement;
receiving at least one medical record associated with the at least one pet from the at least one external pet medical service; and
updating the at least one external pet care service reservation based on the at least one medical record.

10. The computer system of claim 9, the operations further comprising:
associating the at least one external pet care service reservation with the at least one user profile or the at least one pet profile.

11. The computer system of claim 9, wherein creating the at least one external pet care service reservation includes associating the at least one external pet care service reservation with the at least one external pet care service.

12. The computer system of claim 9, the sending the at least one medical record request associated with the at least one pet to the at least one external pet medical service including:
determining at least one pet identifier and the at least one external pet medical service based on the at least one pet profile, wherein the at least one medical record request includes the at least one pet identifier.

13. The computer system of claim 12, wherein the at least one pet identifier includes a pet microchip identifier.

14. The computer system of claim 9, wherein the one or more external pet care services include at least one training pet care service, at least one grooming pet care service, at least one veterinary pet care service, or at least one holistic pet care service.

15. A non-transitory computer-readable medium containing instructions that, when executed by a processor, cause the processor to perform operations for dynamically managing electronic transactions of pet care services data, the operations comprising:
hosting a pet care services portal corresponding to a user and at least one pet, the pet care services portal being based on at least one user profile and at least one pet profile;
receiving pet service search data input by the user via the pet care services portal;
in response to the receiving, determining one or more external pet care services that match the pet service search data;
displaying the one or more external pet care services on a user interface of the pet care services portal;
creating at least one external pet care service reservation in response to a user selection of at least one external pet care service;
in response to creating the at least one external pet care service reservation, determining a medical record requirement for the at least one external pet care service;
sending at least one medical record request associated with the at least one pet to at least one external pet medical service, the at least one medical record request indicating the medical record requirement;
receiving at least one medical record associated with the at least one pet from the at least one external pet medical service; and
updating the at least one external pet care service reservation based on the at least one medical record.

16. The non-transitory computer-readable medium of claim 15, the operations further comprising:
associating the at least one external pet care service reservation with the at least one user profile or the at least one pet profile.

17. The non-transitory computer-readable medium of claim 15, wherein creating the at least one external pet care service reservation includes associating the at least one external pet care service reservation with the at least one external pet care service.

18. The non-transitory computer-readable medium of claim 15, the sending the at least one medical record request associated with the at least one pet to the at least one external pet medical service including:
determining at least one pet identifier and the at least one external pet medical service based on the at least one pet profile, wherein the at least one medical record request includes the at least one pet identifier.

19. The non-transitory computer-readable medium of claim 18, wherein the at least one pet identifier includes a pet microchip identifier.

20. The non-transitory computer-readable medium of claim 15, wherein the one or more external pet care services include at least one training pet care service, at least one grooming pet care service, at least one veterinary pet care service, or at least one holistic pet care service.
